(19) United States
(12) Patent Application Publication
(10) Pub. No.: US 2020/0109006 A1
(43) Pub. Date: Apr. 9, 2020

(54) DIAPER ACCESSORIES

(71) Applicant: Marvoolus Inc., Caledon Village (CA)

(72) Inventors: Matin Vu, Caledon Village (CA); Sara Vu, Caledon Village (CA)

(21) Appl. No.: 16/591,672

(22) Filed: Oct. 3, 2019

Related U.S. Application Data
(60) Provisional application No. 62/740,725, filed on Oct. 3, 2018.

(51) Int. Cl. B65F 1/14; A61F 13/84; B65F 1/16; A47D 5/00

(52) U.S. Cl. CPC B65F 1/1426; A61F 13/84; B65F 1/1638; A47D 5/006; B65F 2240/132; B65F 2210/139; B65F 2210/148; B65F 2210/168; A61F 2013/8402

(57) ABSTRACT

The present application relates generally to diaper accessories, and specifically to changing platforms and disposal bins. The disposal bin of the present application includes an outer housing, the outer housing configured to receive an inner bin and the outer housing including a deposit opening through which a disposal item may be introduced into the outer housing. The disposal bin further includes an inner bin received in the outer housing and defining an inner bin opening for receiving a disposal item when the disposal item is deposited through the deposit opening of the outer housing. The changing platform of the present application includes a core and at least one extension portion being reconfigurable between a collapsed portable configuration and a deployed configuration in which the changing platform is extended to increase the size of a changing surface. A related method of deploying a change table is also described.

### FIELD OF THE INVENTION

The present specification relates generally to diaper accessories, and specifically to changing platforms and disposal bins.

### BACKGROUND OF THE INVENTION

Many people use diapers during at least a period of their lives. For example, for a period of months or years after birth, many infants are dressed in diapers by a caregiver.

The use of diapers often includes performing diaper-related activities. In the case of infants, one such activity is the changing of diapers. For example, when a diaper that is being worn by an infant has been soiled, a caregiver will typically rest the infant on its back on a changing surface in order to ease the activity of removing the soiled diaper, cleaning the infant, and fitting a fresh diaper.

In many cases, a caregiver gives some attention to selecting an appropriate changing surface on which to rest the infant. Often, a caregiver will seek a firm, reasonably comfortable, clean surface for use as a changing surface. Many products have been provided to help in providing an appropriate changing surface. For example, many public restrooms include an installed changing station with the aim of providing a firm and generally horizontal surface. As another example, many diaper bags incorporate a roll-out mat for use in improving the appropriateness of a base surface, such as by providing a barrier between an infant and the base surface to protect the infant from contamination or by providing a thin layer of padding between the infant and the base surface. However, caregivers and others continue to seek improved changing surfaces.

In many cases, when a soiled diaper has been removed it is stored for a period in a living area while the diaper remains in a soiled or partly-soiled state. In the case of reusable diapers, a soiled diaper is often stored for a time before being washed or before a full washing process is complete. For example, a caregiver may remove a soiled diaper, rinse the soiled diaper, and then store the rinsed diaper along with other rinsed diapers in a garage or laundry room until the caregiver has an opportunity to wash an accumulated set of diapers. In the case of non-reusable diapers, a soiled diaper is often stored for a time in a living area until the soiled diaper can be collected by a disposal agent to be removed from the living area. For example, a caregiver may remove a soiled diaper and deposit the soiled diaper in a bin at a home or office or daycare or other living area until a garbage collector can come and collect the soiled diaper.

The caregiver and others will typically seek to store soiled or partially-soiled diapers in a way in which the ambient smell and other unpleasantries associated with the diapers is minimized. Many sealed bins have been disclosed to trap smells and other unpleasantries, but caregivers and others continue to seek improved storage receptacles.

Accordingly, there remains a need for improvements in the art.

### SUMMARY OF THE INVENTION

In an embodiment of the present invention, there is provided a disposal bin, comprising an outer housing, the outer housing configured to receive an inner bin in a lower portion thereof, the outer housing including a deposit opening in an upper portion thereof through which a disposal item may be introduced into the outer housing; and an inner bin, the inner bin received in the lower portion of the outer housing, the inner bin defining an inner bin opening in an open upper end thereof for receiving a disposal item when the disposal item is deposited through the deposit opening of the outer housing.

In an embodiment of the present invention, there is provided a disposal bin, comprising: an outer housing, the outer housing configured to receive an inner bin in a lower portion thereof, the outer housing including a deposit opening in an upper portion thereof through which a disposal item may be introduced into the outer housing; and an inner bin, the inner bin received in the lower portion of the outer housing, the inner bin defining an inner bin opening in an open upper end thereof for receiving a disposal item when the disposal item is deposited through the deposit opening of the outer housing.

According to a further embodiment, the present invention provides a change table, comprising: a core and at least one extension portion, the change table being reconfigurable between a collapsed portable configuration and a deployed configuration in which the at least one extension portion of the change table is extended to increase the size of a changing surface.

According to a further embodiment, the present invention provides a method of deploying a change table, comprising: drawing at least one extension portion away from a core to expose a changing surface of the change table; and deploying a set of legs to support the changing surface above an environmental surface.

Other aspects and features according to the present application will become apparent to those ordinarily skilled in the art upon review of the following description of embodiments of the invention in conjunction with the accompanying figures.

### BRIEF DESCRIPTION OF THE DRAWINGS

The principles of the invention may better be understood with reference to the accompanying figures provided by way of illustration of an exemplary embodiment, or embodiments, incorporating principles and aspects of the present invention, and in which:

FIG. 1 is a perspective view of a disposal bin, according to an embodiment;

FIG. 2 is a left side view of the disposal bin of FIG. 1;

FIG. 3 is a back view of the disposal bin of FIG. 1;

FIG. 4 is a top plan view of the disposal bin of FIG. 1;

FIG. 5A is a perspective view of the disposal bin of FIG. 1, with the inner bin removed to show interior structure;

FIG. 5B is a first perspective view of an upper portion of the disposal bin of FIG. 1, with the deposit door opened;

FIG. 5C is a second perspective view of the upper portion of the disposal bin of FIG. 1, with the deposit door opened;

FIG. 5D is a front view of a disposal bin according to an embodiment, with the deposit door and access door opened;

FIG. 5E is a left side view of the disposal bin of FIG. 5D, with the deposit door and access door opened;

FIG. 5F is a top plan view of the disposal bin of FIG. 5D, with the deposit door and access door opened;

FIG. 6A is a perspective view of the disposal bin of FIG. 1;

FIG. 6B is a perspective view of a locking mechanism of the disposal bin of FIG. 6A;

FIG. 7 is a perspective cut-away view of the disposal bin of FIG. 1;

FIG. 8 is a front elevation cut-away view of the disposal bin of FIG. 1;

FIG. 9 is a left side elevation cut-away view of the of the disposal bin of FIG. 1;

FIG. 10 is a first perspective view of a change table bag, according to an embodiment;

FIG. 11 is a second perspective view of the change table bag of FIG. 10;

FIG. 12 is a third perspective view of the change table bag of FIG. 10;

FIG. 13 is a fourth perspective view of the change table bag of FIG. 10;

FIG. 14 is a perspective exploded view of the change table bag of FIG. 10;

FIG. 15A is a perspective view of a first step in deploying a table of the change table bag of FIG. 10;

FIG. 15B is a perspective view of a second step in deploying the table of the change table bag of FIG. 10;

FIG. 15C is a perspective view of a third step in deploying the table of the change table bag of FIG. 10;

FIG. 15D is a perspective view of a fourth step in deploying the table of the change table bag of FIG. 10;

FIG. 15E is a perspective view of a fifth step in deploying the table of the change table bag of FIG. 10;

FIG. 15F is a perspective view of a sixth step in deploying the table of the change table bag of FIG. 10;

FIG. 16 is a front elevation view of the change table bag of FIG. 10;

FIG. 17 is a perspective view of a change table for use with a change table bag, according to an embodiment;

FIG. 18 is a top plan view of the table of FIG. 17;

FIG. 19 is a side elevation view of the table of FIG. 17;

FIG. 20 is a perspective view of the table of FIG. 17, with the top widened;

FIG. 21 is a top plan view of the table of FIG. 20;

FIG. 22 is a side elevation view of the table of FIG. 20;

FIG. 23 is a perspective view of the table of FIG. 20, with the legs lowered;

FIG. 24 is a top plan view of the table of FIG. 23;

FIG. 25 is a side elevation view of the table of FIG. 23;

FIG. 26 is a perspective view of the table of FIG. 23, with the legs extended;

FIG. 27 is a top plan view of the table of FIG. 26;

FIG. 28A is a perspective view of a change table in a fully deployed configuration, according to an embodiment;

FIG. 28B is a perspective view of the change table of FIG. 28A, in a partially retracted configuration;

FIG. 28C is a perspective view of the change table of FIG. 28A, in a fully retracted configuration;

FIG. 29A is a front view of the change table of FIG. 28A;

FIG. 29B is a front view of the change table of FIG. 28A, in a partially retracted configuration;

FIG. 29C is a front view of the change table of FIG. 28A, in a fully retracted configuration;

FIG. 30A is a side view of the change table of FIG. 28A;

FIG. 30B is a side view of the change table of FIG. 28A, in a partially retracted configuration;

FIG. 30C is a side view of the change table of FIG. 28A, in a fully retracted configuration;

FIG. 31A is a bottom view of the change table of FIG. 28A;

FIG. 31B is a bottom view of the change table of FIG. 28A, in a partially retracted configuration;

FIG. 31C is a bottom view of the change table of FIG. 28A, in a fully retracted configuration;

FIG. 32 is a perspective cut away view of a change table bag according to an embodiment, indicating a fully retracted change table positioned within the change table bag;

FIG. 33 is a perspective view of an inner bin and drop-through door, according to an embodiment;

FIG. 34 is a perspective view of the inner bin and drop-through door of FIG. 33, with forward and rear flaps in an opened position;

FIG. 35 is a top plan view of the inner bin and drop-through door of FIG. 33;

FIG. 36 is a top plan view of the inner bin and drop-through door of FIG. 33, with forward and rear flaps in an opened position;

FIG. 37 is a side view of the inner bin and drop-through door of FIG. 33;

FIG. 38 is a sectional view of the inner bin and drop-through door taken along line A-A of FIG. 37;

FIG. 39 is a side view of the inner bin and drop-through door of FIG. 34; and

FIG. 40 is a sectional view of the inner bin and drop-through door taken along line B-B of FIG. 39.

### DETAILED DESCRIPTION OF THE EMBODIMENTS

The description that follows, and the embodiments described therein, are provided by way of illustration of an example, or examples, of particular embodiments of the principles of the present invention. These examples are provided for the purposes of explanation, and not of limitation, of those principles and of the invention. In the description, like parts are marked throughout the specification and the drawings with the same respective reference numerals. The drawings are not necessarily to scale and in some instances proportions may have been exaggerated in order more clearly to depict certain features of the invention.

An aspect of this description relates to a disposal bin, which includes an outer housing and an inner bin. In some embodiments, an outer housing is a decorative housing, such as a housing designed to look like a bedside table or other common item of furniture. In some embodiments, the disposal bin is a multi-purpose item which includes a changing surface defined by an upper portion of the outer housing, such that the disposal bin may be used also as a changing station. In some embodiments, a changing surface is provided for use as a table to hold a variety of items including an infant or baby supplies or any other items which may normally be held by a table.

In some embodiments, each of an inner bin and an outer housing of a disposal bin independently provide some measure of odour suppression. In some embodiments, an inner bin includes an opening in an upper portion, through which a disposal item may be deposited or removed, and the opening is governed by a drop-through door biased in a closed position such that the drop-through door opens to allow items which drop on the drop-through door to pass through and then closes. In some embodiments, an outer housing encloses the inner bin and defines a deposit opening in an upper portion of the outer housing, through which disposal items may be passed so that they are dropped on a drop-through door of an inner bin. In some embodiments, a deposit opening of an outer housing is governed by an isolation door, provided to reduce the dispersion of odours.

Disposal bins may be used in a variety of settings, such as a home, office, public restroom, daycare or other area where a need to provide a disposal bin for diapers or other disposal items may be expected.

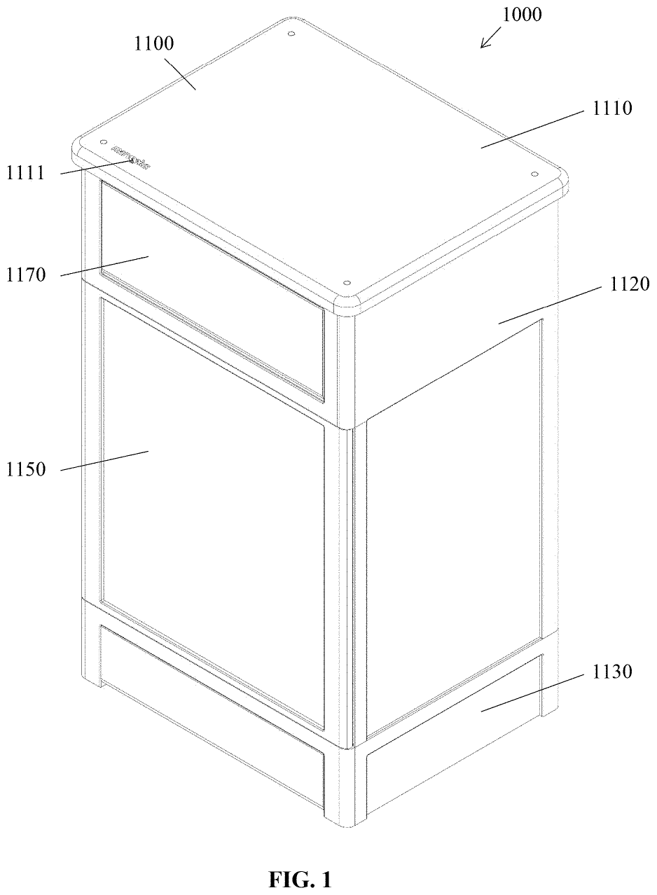

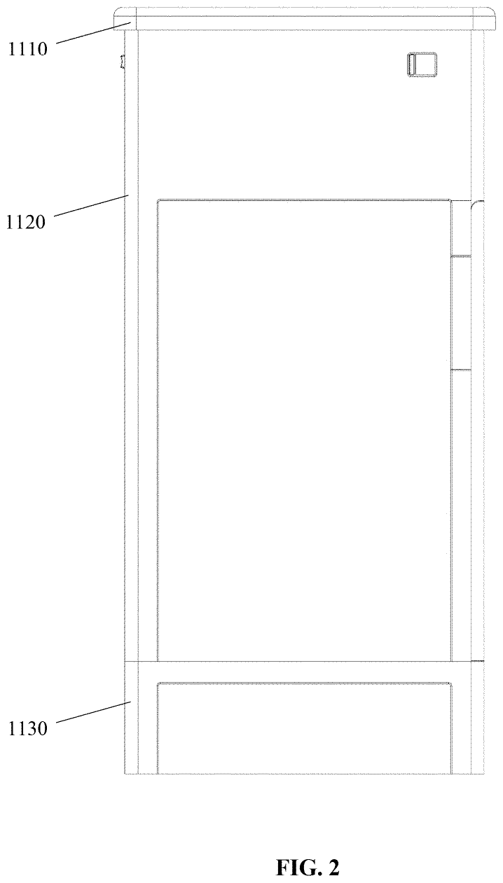

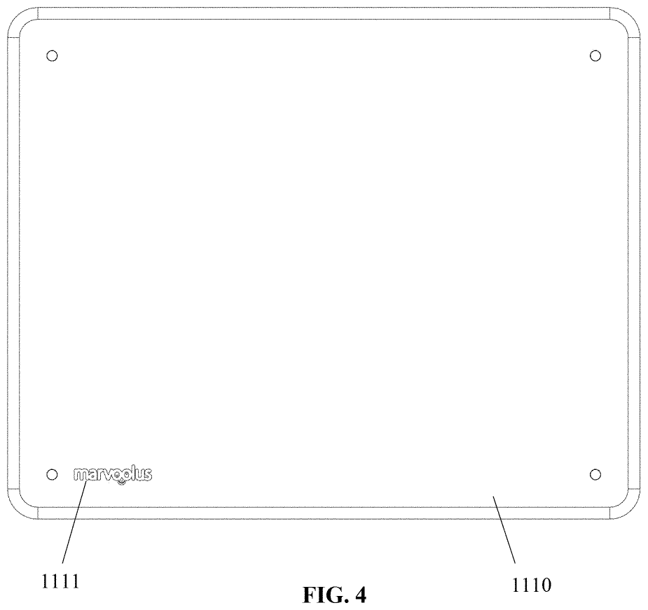

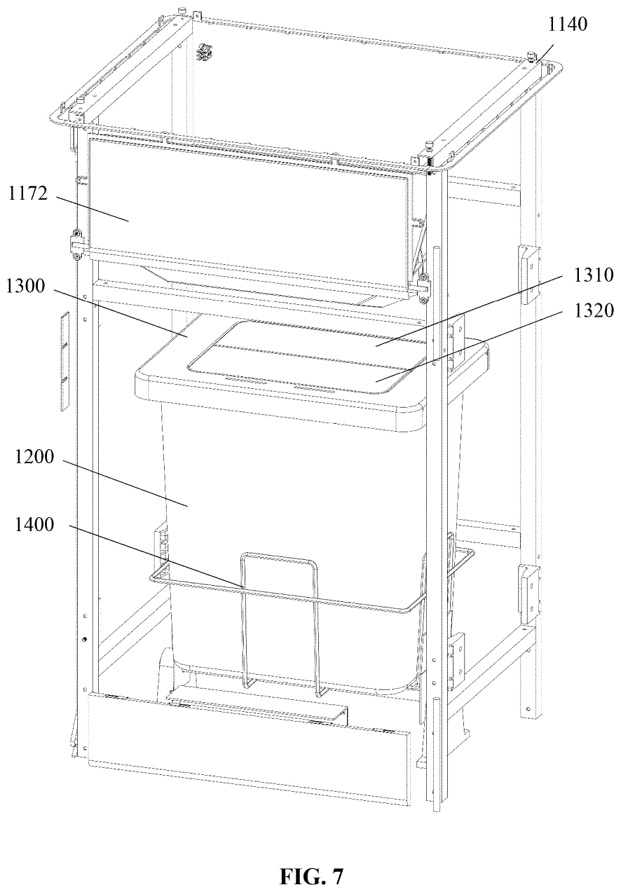

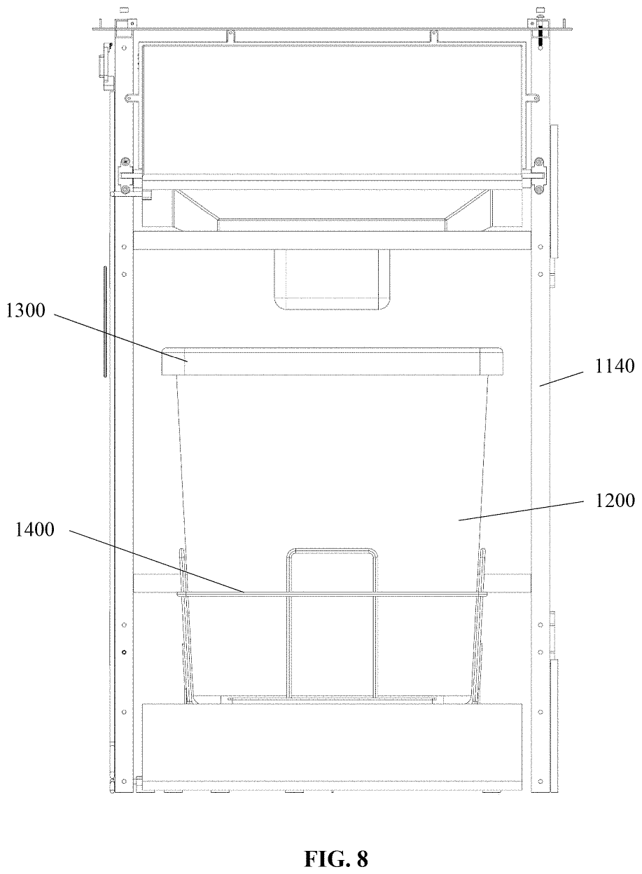

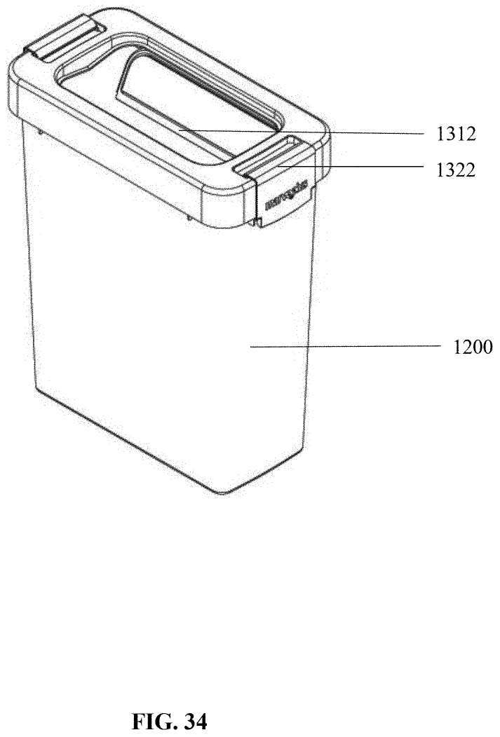

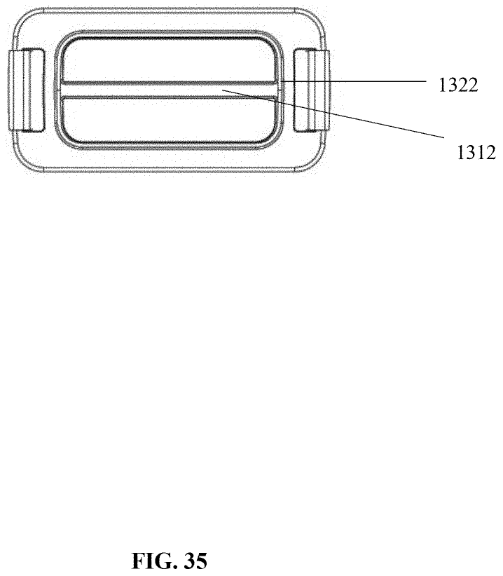

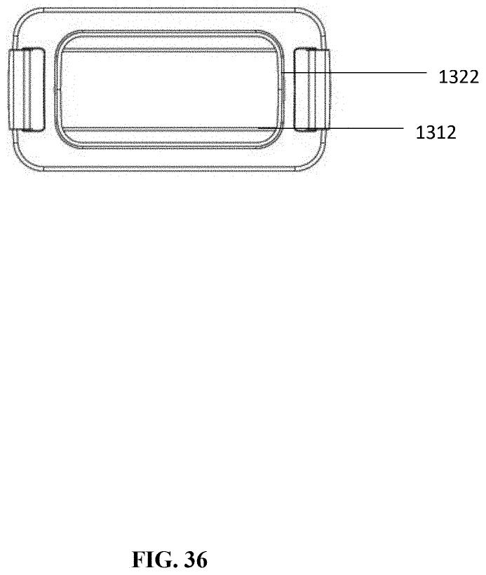

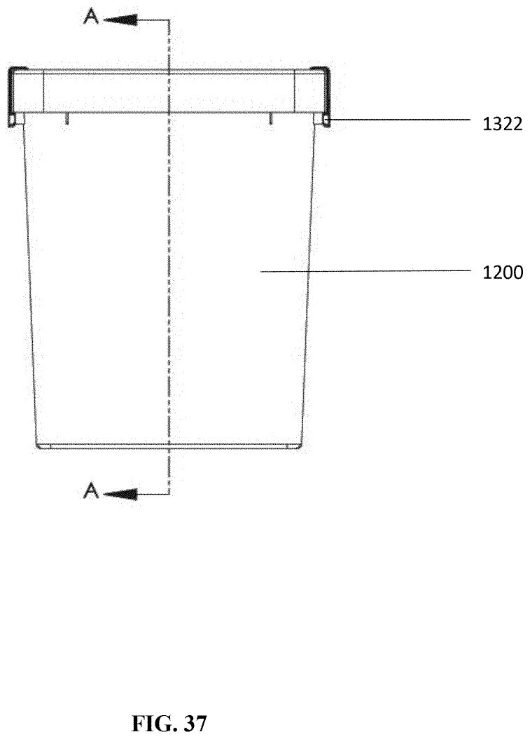

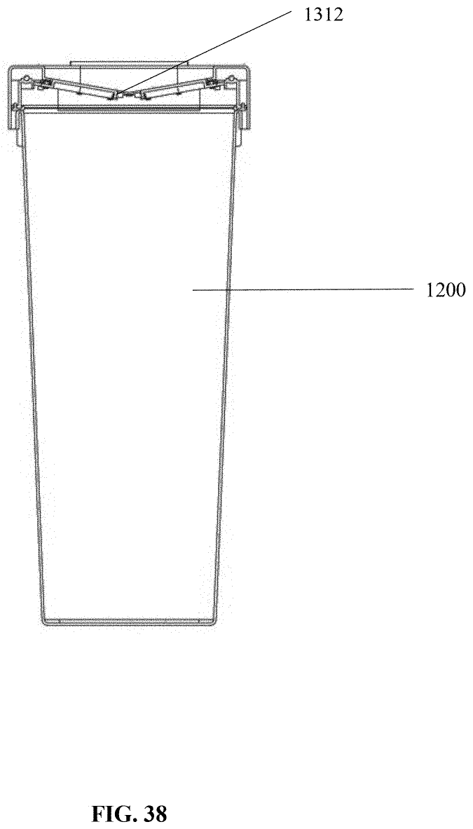

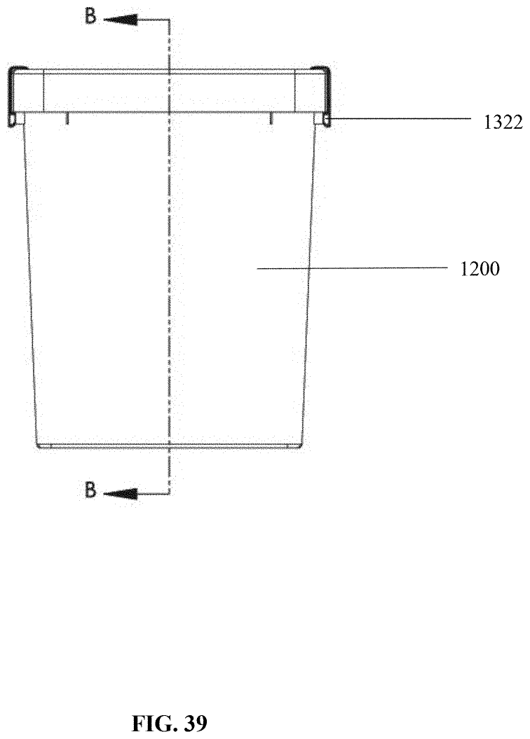

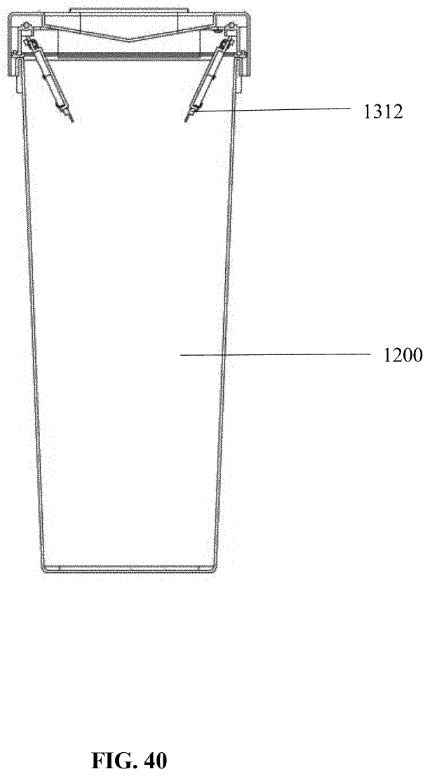

An embodiment of a disposal bin is depicted in FIGS. 1 to 9 and 33 to 40. Disposal bin 1000 includes an outer housing 1100. Outer housing 1100 includes a top 1110, a body 1120, a base 1130, and a frame 1140. Frame 1140, shown particularly in FIGS. 7 to 9, supports the panels that make up the top 1110, body 1120, and base 1130.

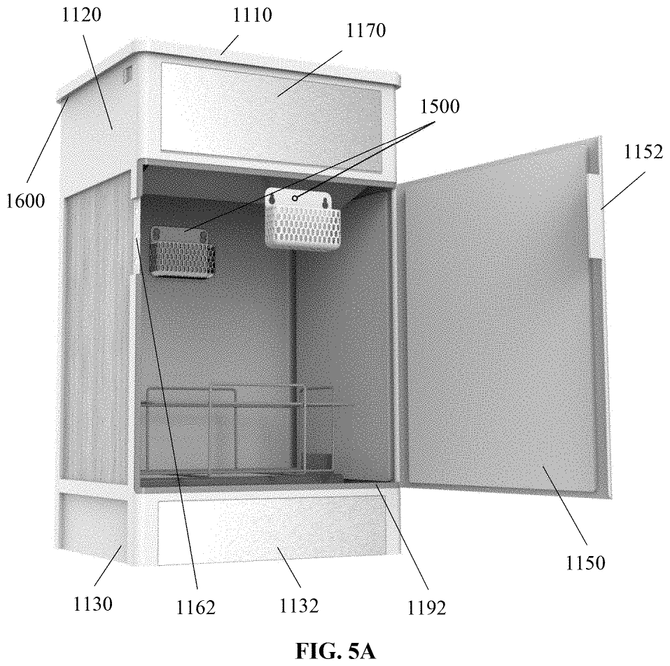

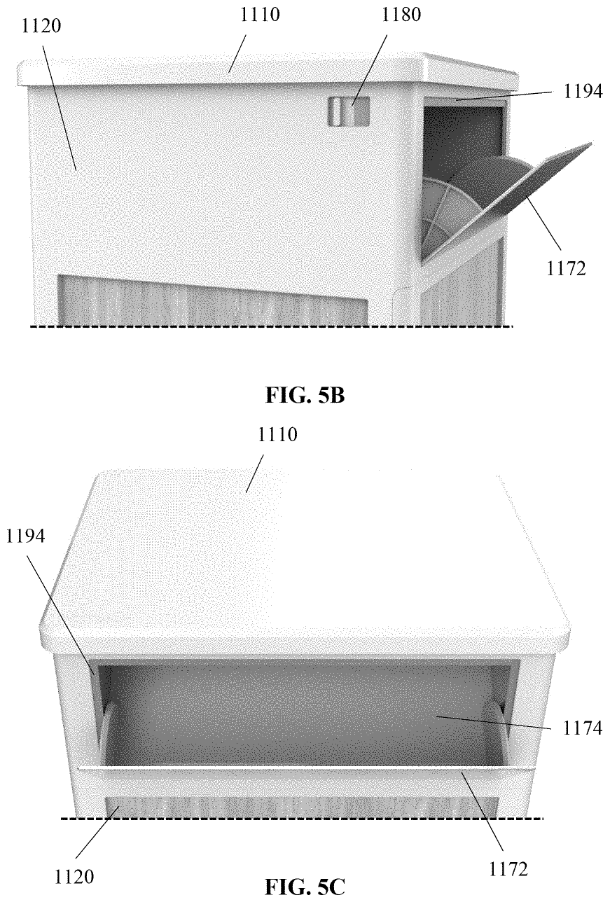

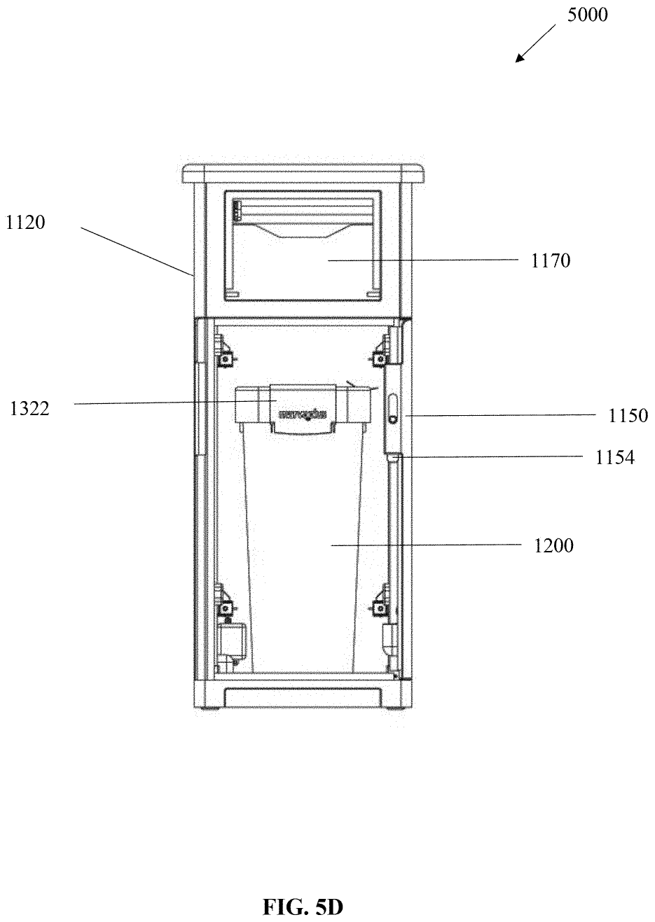

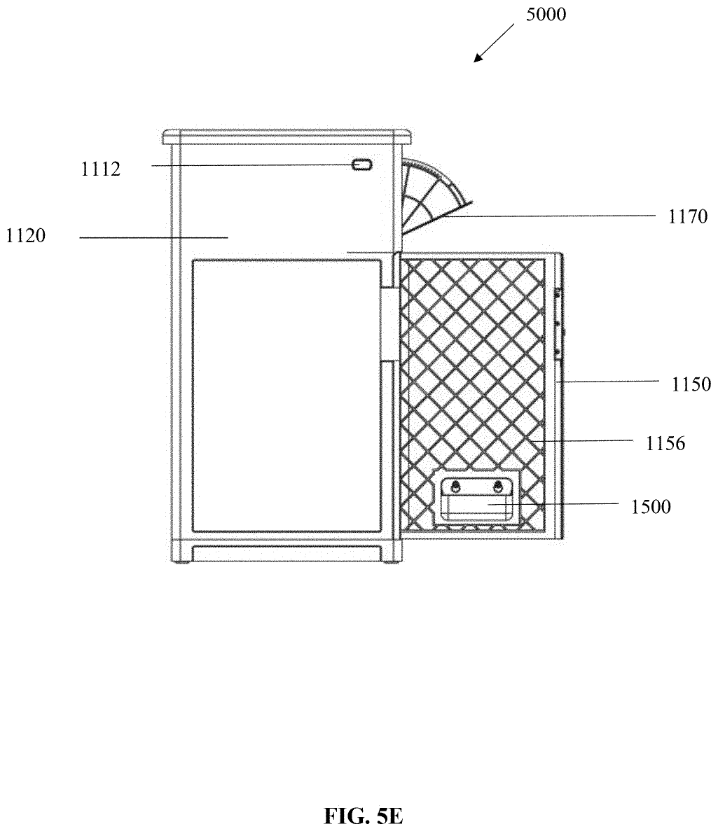

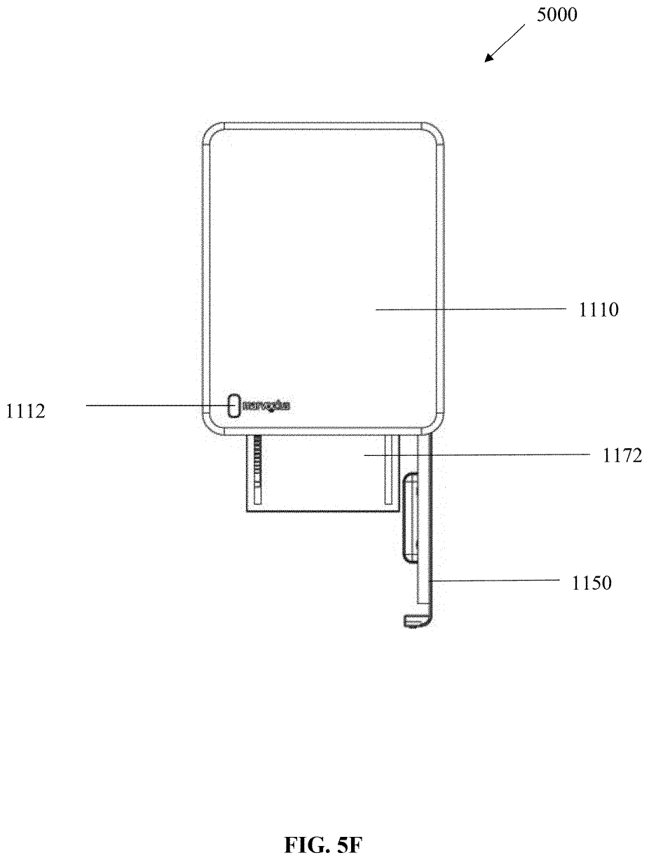

Body 1120 defines an access opening for use in emptying an inner bin, this access opening is governed by an access door 1150. Access door 1150 is hingedly secured to housing 1100, and can be swung open, as shown in FIG. 5A, to allow interior of disposal bin 1000 to be accessed, such as to allow a user to retrieve an inner bin to be emptied. Hinged door 1150 is held in a closed position by a magnetic locking system which includes a first set of magnets 1162 secured to body 1120 and a second set of magnets 1164 secured to hinged door 1150, the first and second sets of magnets 1162 and 1164 being arranged to attract each other when brought into proximity. According to a further embodiment as shown in FIG. 5D, hinged door 1150 may be held in a closed position by a toggle locking system, which includes a toggle 1154 on access door 1150 that can be slid up or down to secure within an opening slot on body 1120. When the toggle locking system is in a closed position, toggle 1154 is disposed within an opening slot on body 1120 to maintain access door 1150 in a closed position. When the toggle locking system is in an open position, toggle 1154 is withdrawn from an opening slot on body 1120 and access door 1150 is unsecured and free to open. Access door may be manually opened by drawing an unhinged edge outward. Access door 1150 includes a handle 1152 for use in opening access door 1150. As indicated in FIG. 5A, handle 1152 is shaped to be received into a portion of the paneling of body 1120 so that it is discreetly integrated and less noticeable, such as to assist in keeping it from becoming a target of interest for an infant or young child. Magnets may be, for example, neodymium magnets. In some embodiments, access door 1150 may be hinged via a latch or snap lock system.

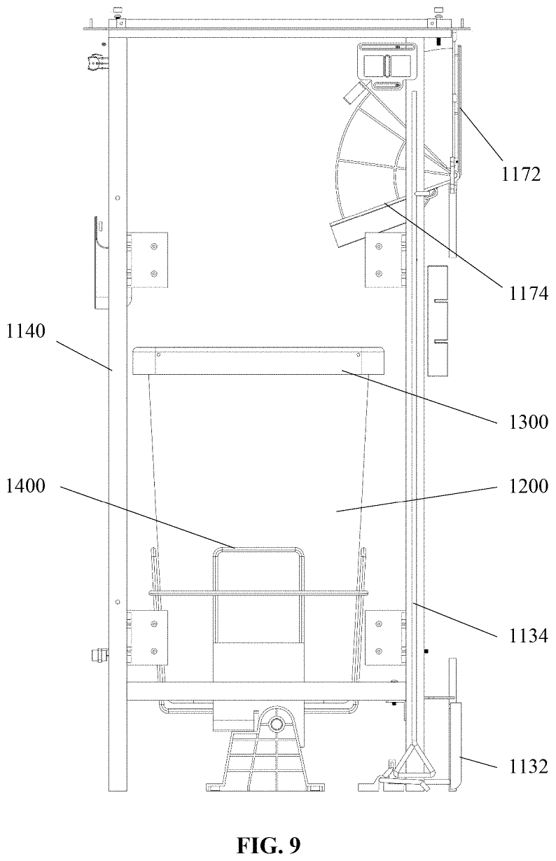

Body 1120 also defines a deposit opening in an upper portion for receiving a deposit of a disposal item. To minimize the dispersion of odours during a deposit, this deposit opening is governed by isolation door 1170. Isolation door 1170 is pivotably secured to the housing 1100. Isolation door 1170 is biased in a closed position and may be opened by operation of a foot pedal 1132 formed in base 1130 and connected to isolation door 1170 via shaft 1134, as shown particularly in FIG. 9. A foot pedal allows a caregiver, whose hands may be dirty, to easily dispose of an item.

In some embodiments, such as is shown in FIGS. 5E and 5F, an electronic wave sensor or motion sensor or other sensor is located on a disposal bin and may be activated to trigger an action such as the opening of an isolation door. For example, in some embodiments a disposal bin includes a wave sensor 1112 so that a caregiver can gesture their hand or body part in front of the sensor to trigger an electronic control system to open isolation door 1170; providing a “no-touch” opening option. The wave sensor may also be disabled by, for example, toggling a switch that disconnects a circuit to stop electric current from flowing to the sensor. This switch may be located inside body 1120 under isolation door 1170. Disabling this switch prevents the electronic motion sensor from being accidentally triggered, thereby preventing isolation door 1170 from opening. An electronic wave sensor or motion sensor or other sensor may be placed anywhere that may allow for convenient triggering of the sensor, such as on top 1110 or body 1120 or both. In some embodiments a sensor replaces physical handles, while in other embodiments which include sensors the sensors are provided in addition to physical handles.

The isolation door 1170 forms a forward panel 1172 and a rear panel 1174 fixedly joined along a bottom edge to form a v-shaped deposit area. When isolation door 1170 is closed, as shown in FIG. 5A, the forward panel 1172 is held against an outer surface of body 1120 to provide a barrier to the free movement of air in and out of the deposit bin 1000. Isolation door 1170 may also be opened to allow a user to deposit a disposal item, such as a diaper. Isolation door 1170 may be opened by pivoting the forward panel 1172 out and down, which causes the rear panel 1174 to also move, such that when the isolation door is open the rear panel provides a barrier to the free movement of air in and out of the deposit bin. When a disposal item has been placed in the v-shaped deposit area and the deposit door 1170 is closed, the rear panel 1174 is sufficiently included to allow a disposal item to fall out of the v-shaped deposit area and into a lower portion of the outer housing 1100. Rear panel 1174, when in a closed position, also acts to define a path to direct the disposal item into the middle of the inner housing, as indicated in FIG. 9. Lock engagement toggle 1180 may be manipulated to engage or disengage a locking feature which allows a user to lock deposit door 1170 in a closed position.

As depicted, disposal bin 1000 defines a flat top, which may be used as a changing surface for changing the diaper of an infant or simply as a table to hold items such as baby supplies or any other items which may normally be held by a table. As depicted, the top of bin 1000 is a flat smooth surface save for a logo 1111 inscribed near an edge. Top 1110 may also contain an electronic wave sensor or motion sensor or other sensor 1112. However, in some embodiments the disposal bin may include further features to ease the use of the disposal bin as a changing station, such as a raised perimeter around the edge of a changing surface, an integrated speaker to play music, a projector located on top 1110 to project images onto a wall or ceiling surface, a USB port located on disposal bin 1000 for a caregiver to be able to download files so as to project their desired images, videos, or sounds from the disposal bin, an integrated concealed warming/heating pad or element located on top 1110 so that a caregiver can heat up liquid such as milk or baby wipes to a warmer temperature or a wireless charging pad on top 1110 so that a caregiver may charge a device.

Housing 1100 also includes a set of seals to assist in sealing in odours. The set of seals includes an access opening seal 1192, a forward deposit opening seal 1194, and a rear deposit opening seal (not shown). When access door 1150 is closed, it abuts access opening seal 1192 to help in blocking the dispersion of odours through the access opening. When deposit door 1170 is closed, it abuts forward deposit opening seal 1194 to help in blocking the dispersion of odours through the deposit opening. When deposit door 1170 is opened, rear panel 1174 abuts a rear deposit opening seal (not shown) to help in blocking the dispersion of odours through the deposit opening. Seals may be any of a variety of materials which are effective in helping to block the dispersion of odours, such as semi-malleable plastic or rubber strips held to the respective panels or items by adhesive. For example, a silicone gasket may be provided along all joins in the housing 1100.

Disposal bin 1000 also includes an inner bin 1200. The inner bin 1200 is a moulded bin defining a storage opening in an upper portion. Inner bin 1200 is a storage bin for holding disposal items such as diapers that have been deposited through the deposit opening of the outer housing 1100, directed to the middle of outer housing 1100 by rear panel 1174, and fallen down through the middle of outer housing 1100 and into the inner bin 1200 through the storage opening. The inner bin 1200 is configured for use with standard garbage bags as liners for ease in emptying the inner bin. Inner bin 1200 is also substantially cubical rather than cylindrical to ease the removal of a liner from the inner bin 1200; often a garbage bag or other liner of a bin can be difficult to remove from a cylindrical bin as the cylindrical shape does not provide appropriate air channels, resulting in a suction effect when a user tries to draw out the liner. The large cubical shape of the inner bin 1200 also facilitates its use as a laundry hamper or garbage bin later in a bin lifecycle. In some embodiments, an inner bin has surface features such as dimples, ridges or grooves moulded on an inside surface of the sides of the inner bin to improve air flow around a liner and further reduce the suction effect when pulling out a liner.

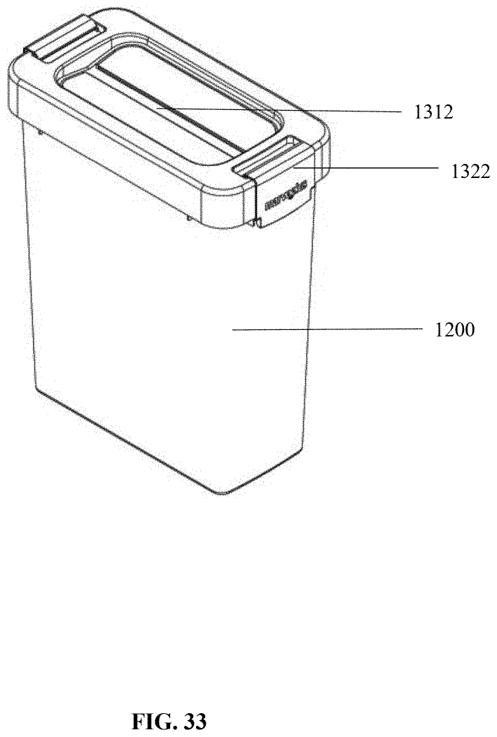

The storage opening defined in an upper portion of inner bin 1200 is governed by a drop-through door 1300 in a trap-door style. Drop-through door 1300 includes a forward flap 1310 and a rear flap 1320. Forward and rear flaps 1310 and 1320 are biased in a closed position in which they are raised up against a rim of door 1300. The biasing system holding flaps 1310 and 1320 is fixed with a biasing force that allows the flaps to pivot downward along an outer edge of the flaps when a disposal item such as a diaper falls on the flaps. The drop-through doors allow disposal items to fall through the door and into the inner bin, at which point the biasing force pushes the flaps closed again to block the dispersion of odours via seals 1312 around the perimeter of each flap (seen in FIGS. 33 to 36, 38 and 40).

Drop-through door 1300 includes a sealing element at an interface point or area to assist in sealing odour within inner bin 1200. Drop-through door 1300 is held to the inner bin via a friction fit. Drop-through door 1300 sits on a seal (not shown) on the top perimeter of inner bin 1200 to assist in sealing odour within the inner bin. Forward and rear flaps 1310 and 1320 abut seals 1312 (seen in FIGS. 33 to 36, 38 and 40) when in a closed position under the perimeter of the drop-through door opening to help in blocking the dispersion of odours. According to a further embodiment, drop-through door 1300 may include a seal (not shown) around the bottom perimeter of drop-through door 1300, which when placed on top of inner bin 1200, the two components secure to one another via a friction fit to create a secure seal. In other embodiments, mechanical or other fasteners may be used, such as snaps or hook-and-loop fasteners or adhesives. For example, according to a further embodiment as shown in FIG. 5D, and 33 to 40, drop-through door 1300 may include two snap lock handles 1322 instead of, or in addition to a friction fit, on each end of a drop-through door, which can securely clasp onto inner bin 1200.

In some embodiments, only one of an inner bin and an outer housing is sealed or is extensively sealed. In an example embodiment, an outer housing is sealed using silicon gaskets at all joins in the housing, and the inner bin is an open-topped inner bin with no lid or with a loosely-fitted lid. In another example embodiment, the inner bin is a sealable bin and a lid of the inner bin is tightly secured to the inner bin and is substantially air-tight, such as a drop-through lid as described in reference to disposal bin 1000, and the corresponding housing does not include an isolation deposit door or does not include seals around the deposit door or the access door. However, in preferred embodiments both the inner bin and the outer housing are substantially airtight.

In some embodiments, an outer housing of a disposal bin is interchangeable so that a caregiver or other user can replace it to match a given set of décor. In some embodiments the outer housing may be used with an inner laundry hamper or other inset inner bin, regardless of whether the inner bin is for use with diapers or other odourous disposal items. In some embodiments, proportions of a disposal bin are larger then necessary for a diaper to allow the bin to be used for other items either instead of diapers or after a user no longer needs a diaper disposal bin; for example, a deposit opening and a deposit door may be sized to receive items substantially larger than an average diaper.

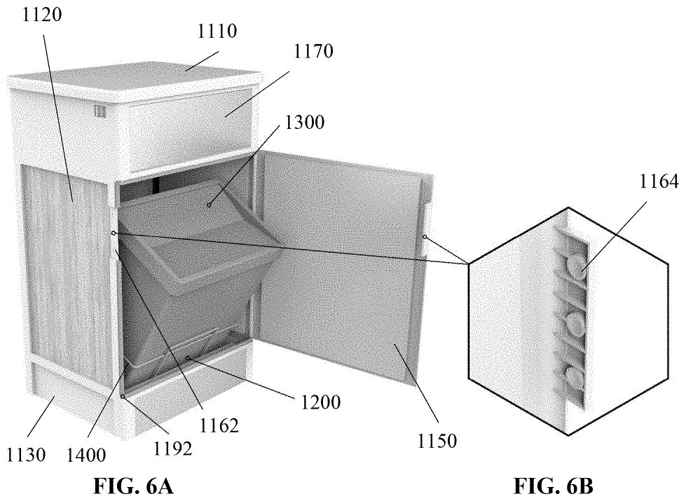

Disposal bin 1000 may also include a presentation basket 1400. Presentation basket 1400 is pivotally secured to frame 1140 and is provided to receive the inner bin 1200. Presentation basket 1400 is proved to ensure that the inner bin 1200 is secured generally in the middle of outer housing 1100 so that disposal items will fall onto the drop-through door 1300. Presentation basket 1400 is pivotally secured to frame 1140 so that it can be pivoted forward as depicted in FIG. 6A. Pivoting inner bin 1200 forward allows a user to access the inner bin 1200 without needing to lift the inner bin 1200 out of the outer housing 1100, such as to remove a garbage bag or other liner from the inner bin. Basket 1400 rests on a pivot point or pivoting surface, and can be drawn into a pivoted position mechanically by a user. In some embodiments a pedal or other actuation element may be provided for use in causing basket 1400 to pivot. In some embodiments, presentation basket 1400 may not be included, as is shown in FIG. 5D.

Disposal bins may also include a variety of additional features for use in reducing or covering the odours released by the disposal bin. For example, disposal bin 1000 also includes a scent pocket 1500. Scent pocket 1500 may be secured to the frame 1140 of the outer housing 1100 and is provided to receive scent-releasing or scent-absorbing inserts for use in overcoming or absorbing odours of the disposal bins. According to a further embodiment as shown in FIG. 5E, scent pocket 1500 may be secured to the inner portion of access door 1150. Scent pocket 1500 is removable and dishwasher safe.

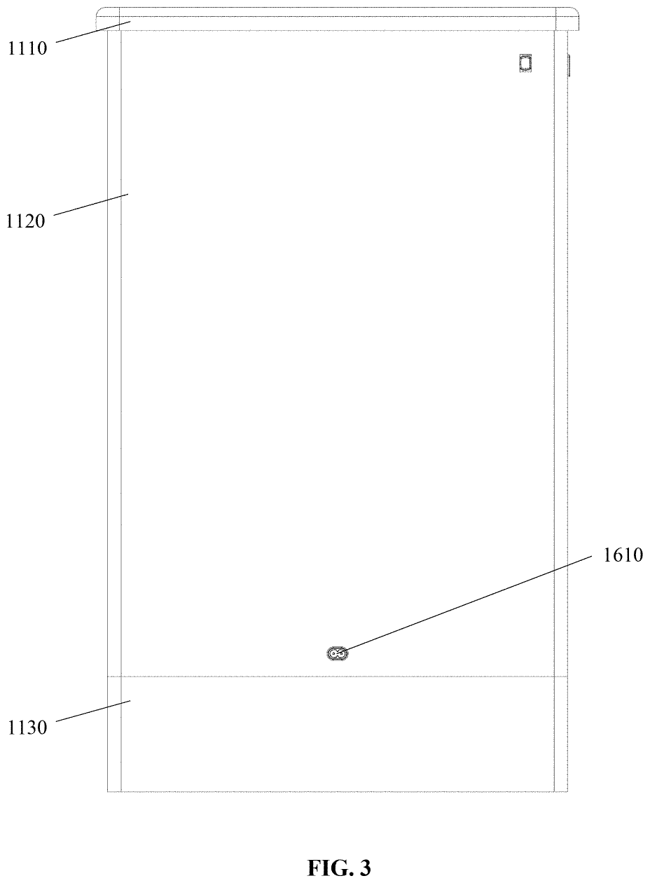

Disposal bin 1000 also includes an ambient lighting element 1600 deposited as a lighting strip under a lip of top 1110, as depicted particularly in FIG. 5A. Ambient lighting element 1600 does not provide lighting directly on the top 1110 of disposal bin 1000 as such could disturb an infant, but provides soothing environmental lighting to permit a caregiver to navigate and operate. Lighting element 1600 is a warm light emitting diode (LED) lighting strip received in a perimeter recess under the lip of top 1110 and covered by a frosted plastic insert to separate the LED strip from the environment. Power for the lighting element or any other powered element of disposal bin 1000 is provided to disposal bin 1000 via a plug 1610, as shown in FIG. 3. Plug 1610 may be for example a DC barrel jack connector, and may be elevated to avoid contact with a baseboard or other environmental feature. In some embodiments, an electronic wave sensor or motion sensor or other sensor may be located on a disposal bin and may be activated to trigger an action such as turning on ambient lighting element 1600. The wave sensor may also be disabled by, for example, toggling a switch that disconnects a circuit to stop electric current from flowing to the sensor. This switch may be located inside body 1120 under isolation door 1170. Disabling this switch prevents the electronic motion sensor from being accidentally triggered, thereby preventing ambient lighting element 1600 from turning on. An electronic wave sensor or motion sensor or other sensor may be placed anywhere that may allow for convenient triggering of the sensor, such as on top 1110 or body 1120 or both.

A variety of materials may be used to form the disposal bin, including metals, plastics, and woods. For example, a frame may be formed of aluminum and may hold up plastic or wood panels making up a top, body, and base of an outer housing. Various panels or components of an outer housing or inner bin may be held together mechanically, such as by means of screws or nails, may be held together via an adhesive, or may be moulded together. In some embodiments, adhesive is preferred over mechanical fastening as an adhesive provides some inherent measure of odour sealing, while mechanical fastening may require more seals to be applied between panels or components.

In some embodiments, one or more components of a disposal bin are formed of an acrylonitrile-butadiene-styrene (ABS) and polycarbonate plastic blend. In some embodiments, one or more components of a disposal bin are formed of stainless steel or aluminum. In some embodiments, as shown in FIG. 5E, one or more components of a disposal bin include features that enhance structural integrity, such as ribbing and gussets 1156 on access door 1150 or outer housing 1100 or both. In some embodiments, one or more components of a disposal bin are formed of vinyl or are covered by peel-and-stick vinyl graphics to allow the look of the bin to be easily modified by replacing a panel or a graphic. For example, a disposal bin may have an aluminum frame supporting ABS or ABS and polycarbonate top and body panels and a brushed stainless steel forward panel of a deposit door and a base which includes brushed stainless steel panels. In some embodiments, a foot pedal provided as part of a base of a disposal bin and provided to control the opening of a deposit door may, in particular, be formed of a durable material such as stainless steel. In some embodiments, an inner bin is formed of a durable firm plastic. Smooth materials are used in some embodiments, such as to provide easily cleaned surfaces. In some embodiments, outer housing may be comprised of individual panels or as a single molded structure form.

An aspect of this description relates to a change table bag, which incorporates a change table and a diaper bag in a practical and portable form. In some embodiments, the change table bag includes a changing table and a wall system forming at least one compartment. In some embodiments the change table bag is reconfigurable between a bag configuration and a change table configuration.

In some embodiments a change table is received within a wall system of a change table bag and is only exposed when the change table bag is reconfigured into a change table configuration. In some embodiments, a change table forms a part of the outer surface of the change table bag when the change table bag is in a closed configuration.

In some embodiments, a change surface defined by a change table of a change table bag is part of the exterior surface of the change table bag when in a bag configuration. However, in other embodiments a change surface defined by a change table of a change table bag is at least partially covered by the wall system when the change table bag is in a bag configuration, to be exposed for use during a diaper changing operation when the change table bag is in a change table configuration.

In some embodiments, the change table bag includes a change table and a wall system, the wall system forming a forward component and a rear component sandwiching the change table therebetween when the change table bag is in a bag configuration and each component being at least partially detachable from the change table to expose a face of the change table. In some embodiments, the change table is an expandable change table, which can be collapsed to reduce bulk and expanded for use as a changing surface.

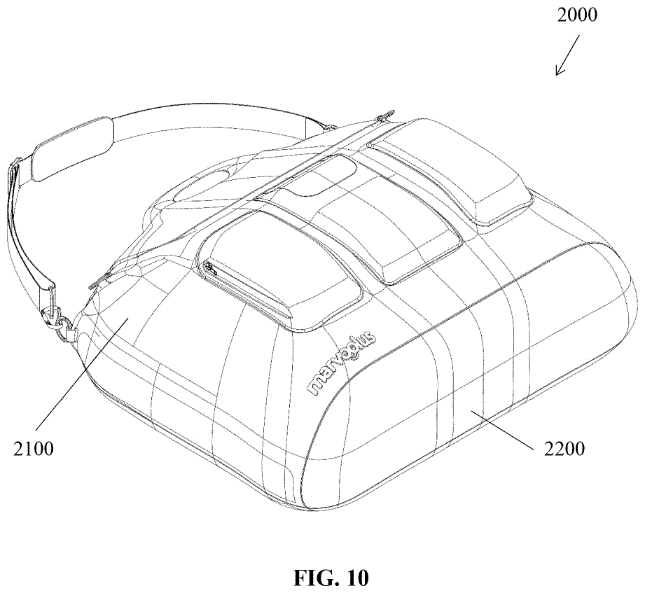

An embodiment of a change table bag is depicted in FIGS. 10 to 16. Change table bag 2000 includes a wall structure 2100 and a change table 2200. Wall structure 2100 defines a forward component 2110 and a rear component 2120 between which the change table 2200 is sandwiched when the change table bag 2000 is in a bag configuration.

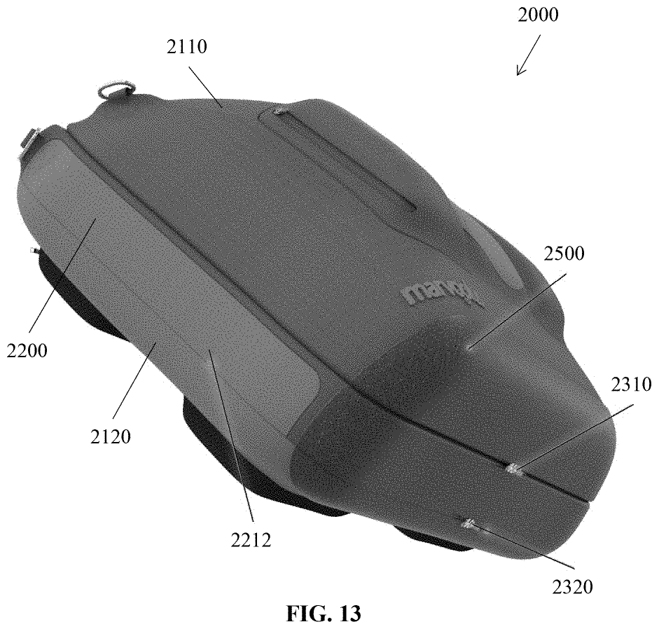

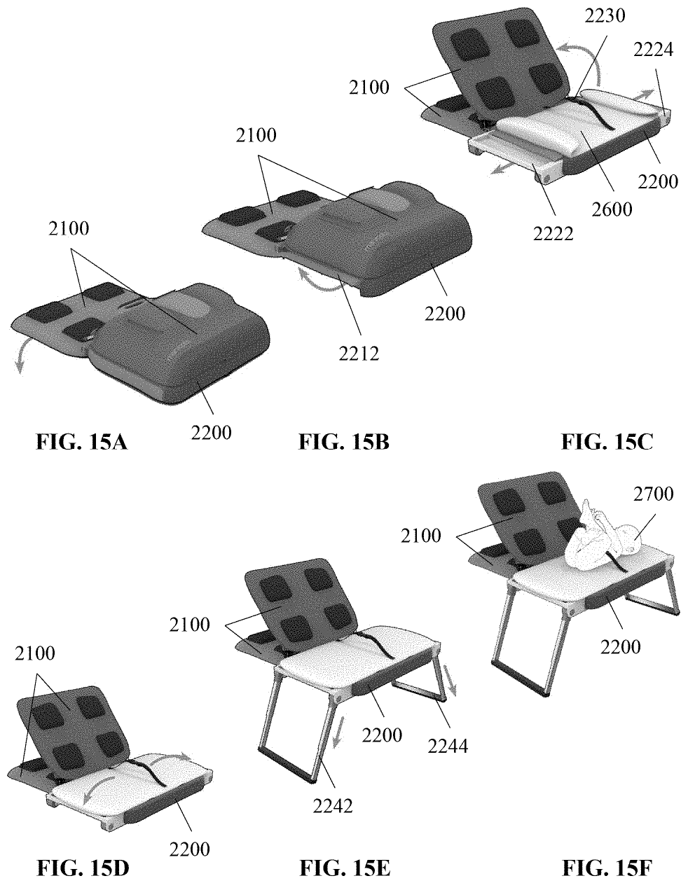

Each of forward and rear components 2110 and 2120 is removably securable to the change table 2200 by a releasable fastener, the releasable fasteners being zipper 2310 and 2320, respectively, shown for example in FIG. 13. Change table 2200 provides a change surface on an upper face of change table 2200. As indicated, in FIGS. 15A to 15F, zippers 2310 and 2320 permit a user to detach or partially detach the wall structure 2100 from the change table 2200 to expose an upper surface of change table 2200 for use as a change surface, and to expose a lower surface of change table 2200 to allow a user to deploy change table support structure.

Change table bag 2000 is configured for use as a diaper bag, with multiple compartments for the storage of spare diapers and other related items. However, in other embodiments, a change table bag could be for other purposes, such as to store or carry items that are not related to diaper changing. The change table can also be used for other purposes such as a table surface for uses other than to change a baby, such as to place a laptop or to use as a table surface for eating or for playing or writing on.

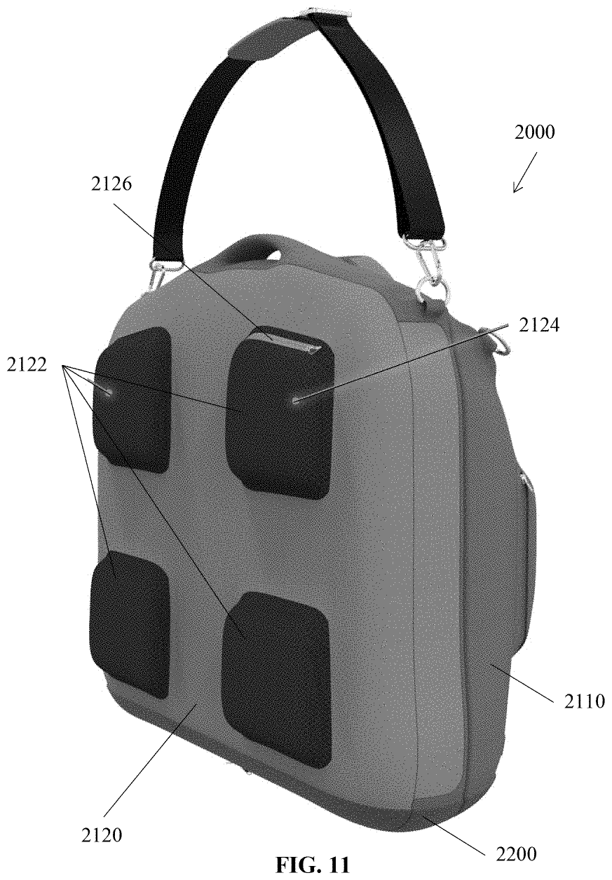

Rear component 2120 defines a set of pads 2122, provided for improved comfort in carrying the change table bag 2000. Pad 2124 of the set of pads 2122 forms a hidden compartment accessible through an opening governed by hidden compartment zipper 2126, as shown particularly in FIG. 11. Rear component 2120 is provided to cover a lower surface of change table 2200, and may be used for any of a variety of reasons including one or more of comfort, protection, sanitation, or aesthetics. In some embodiments, a rear component may also define a storage compartment.

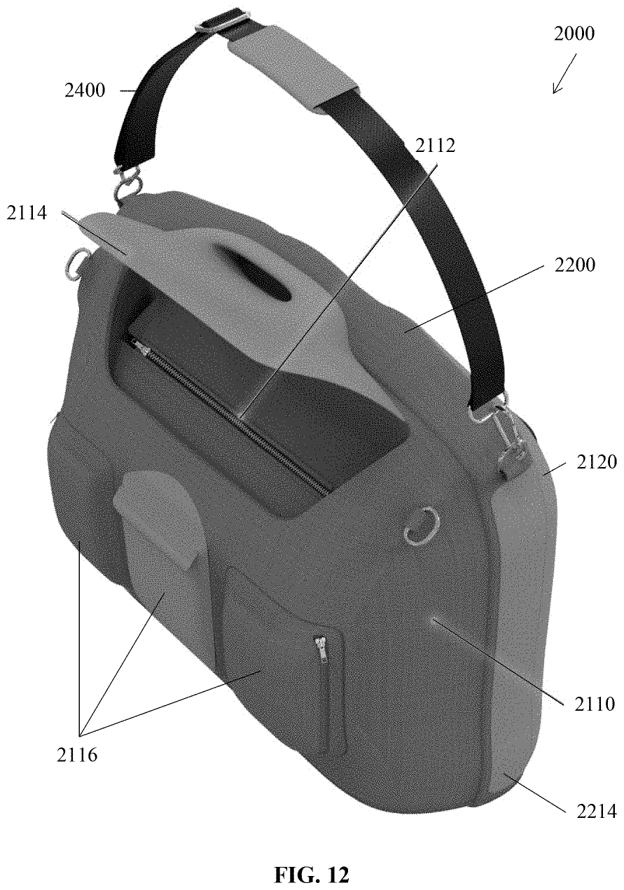

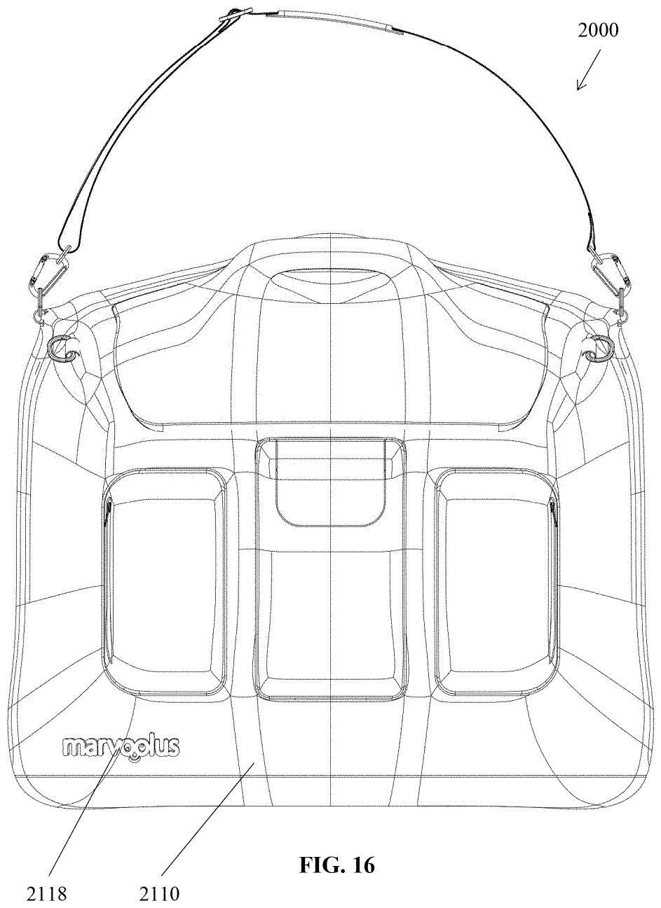

Forward component 2110 defines a main forward storage compartment enclosed by part of the wall structure 2100, and accessed through an opening governed by zipper 2112, shown particularly in FIG. 12. Zipper 2112 is itself covered by flap 2114, which may be raised by a user to access zipper 2112. When it is closed, as shown for example in FIG. 16, flap 2114 may be held in a closed position by a system of magnets, the system of magnets including one or magnets secured to the flap 2114 attracted to one or more magnets secured to a body portion of forward component 2110. Flap 2114 also defines a handle thereon for use in carrying change table bag 2000 independently of any handle included as part of the change table 2200.

In some embodiments, zipper 2112 is replaced by a magnetic closure system, while flap 2114 is held by a zipper rather than a magnetic closure system. In various embodiments, various releasable fasteners may be interchangeable with other releasable fasteners. For example, in some embodiments magnets may be preferred as presenting fewer rough edges, while in some embodiments zippers may be preferred as providing a more uniform closure or a mechanically stronger closure.

Forward component 2110 includes a set of three front compartments 2116 for additional storage capacity, as indicated in FIG. 12. The set of front compartments 2116 includes two flanking zipper-access diaper or storage pouches and one center baby bottle pouch. Forward component 2110 also includes an embroidered logo 2118.

A change table bag may also include a system of straps or other carry aids. Change table bag 2000 includes a shoulder strap 2400. Shoulder strap 2400 is secured to edges of change table 2200. In some embodiments, straps or other carry aids may be secured to a wall structure of a change carry bag. In some embodiments, carry aids may include backpack-style straps or other straps or handles or carry aids. Straps may be detachable or movable from one attachment point to another. In some embodiments, straps or other carry aids may contain a storage pouch stitched within it to hold electronics or other devices.

In some embodiments, one or more storage components may include an internal organizer to allow a user to keep items in order and accessible. For example, in some embodiments which include a forward component defining a main forward compartment, the main forward compartment will include an internal organizer, such as a system of pouches or internal walls. In some embodiments, one or more storage components may include a built-in battery pack for wireless charging of devices or a heating element to warm up or keep warm foods or liquids or both.

Wall structure 2100 is made of a durable, lightweight canvas. In various embodiments, a wall structure may include a variety of different canvases or fabrics or plastics or other durable materials suitable for use in forming diaper bags. In some embodiments, a wall structure is water retardant and stain resistant and may be made of rubber. In some embodiments, a wall structure includes antibacterial materials or other sanitation elements.

In some embodiments, part of a wall structure or parts of both a wall structure and a change table are configured for use as a bottom or base of the change table bag. For example, change table bag 2000 includes a base 2500 formed of an extra layer of plastic, leather, or rubber coating or durable fabric on a base portion of each of forward component 2110, rear component 2120, and change table 2200, as shown particularly in FIG. 13. In some embodiments, the perimeter, top or side of the change table bag 2000 may contain a built-in light to provide ambient walkway lighting or direct spot lighting for safety and security.

As shown particularly in FIGS. 15A to 15F, in reconfiguring change table bag 2000 from a bag configuration to a change table configuration a number of steps may be employed. As shown in FIG. 15A, a user may release zipper 2320 to partially detach rear component 2120 from change table 2200, and may then swing rear component 2120 away from a rear surface of change table 2200. As shown in FIG. 15B, the user may then lift left panel 2212 and right panel 2214 to permit the free movement of an extension portion of change table 2200. Panels 2214 and 2212 are shown, respectively, in FIGS. 12 and 13. Extension portions are provided to allow the change table to conform to a standard change surface size when extended, while providing a more compact profile for portability.

As shown in FIG. 15C, the user may then draw out left extension portion 2222 and right extension portion 2224. The user may also release zipper 2310 to partially detach forward component 2110 from change table 2200, and may then swing forward component 2110 away from a forward surface of change table 2200. As depicted in FIG. 15C, a mat 2600 is used to cover the forward surface of change table 2200. In some embodiments, a mat may be secured or partially secured to change table 2200, while in some embodiments no mat is needed or a mat is incorporated into change table 2200. Change table 2200 also includes a strap 2230 for use in holding an infant on the change table 2200 during a diaper changing operation.

As shown in FIG. 15D, mat 2600 may be deployed to cover the forward surface formed by change table 2200 after extension portions 2222 and 2224 are drawn out. As shown in FIG. 15E, the user may then deploy a change table support structure, the change table support structure including left leg 2242 and right leg 2244. As shown in FIG. 15F, the user may then rest an infant 2700 on the mat 2600 for a diaper change operation.

Change table bag 2000 may also be further reconfigured to completely remove the change table 2200 by fully unzipping front portion 2110 and rear portion 2120 from the change table and then rezipping front portion 2110 and rear portion 2120 together without change table 2200 between. A set of anchor points for strap 2400 are provided on front portion 2110 to allow the reconfigured bag to be carried via strap 2400 even without the change table 2200. The change table can also be carried separately without the wall system 2100, since the change table 2200 can be detached from the wall system 2100 and since the change table 2200 includes a handle and anchor points for strap 2400.

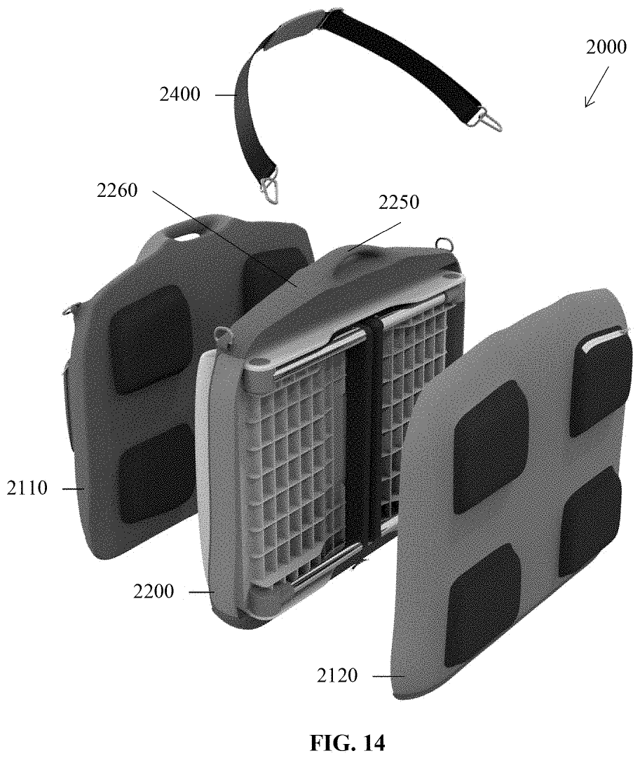

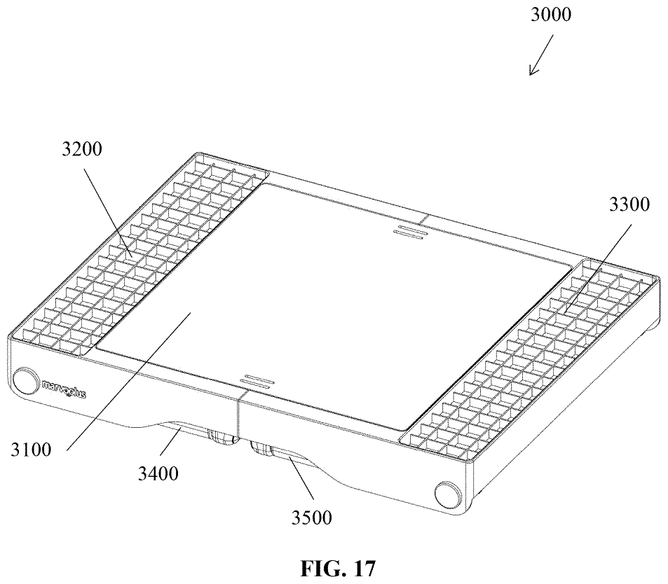

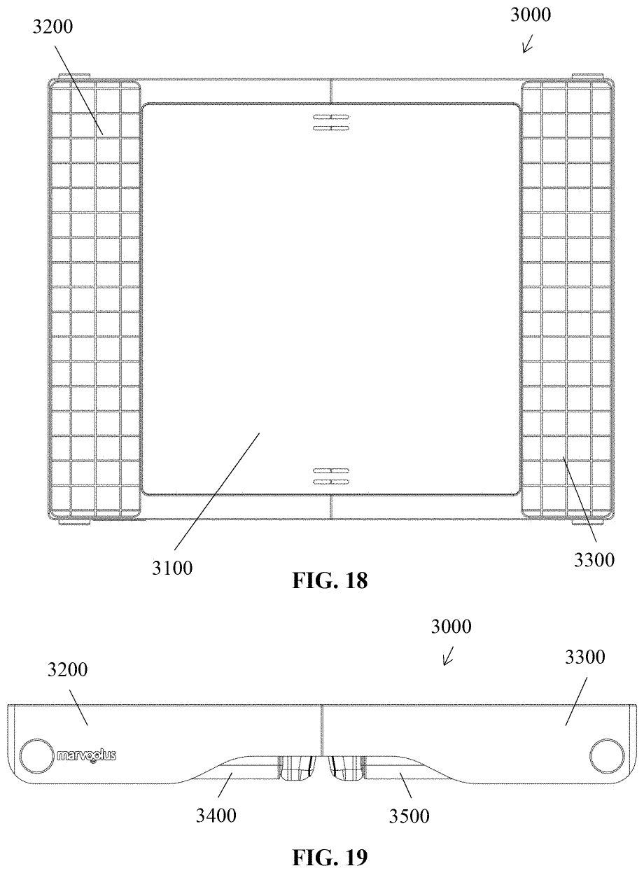

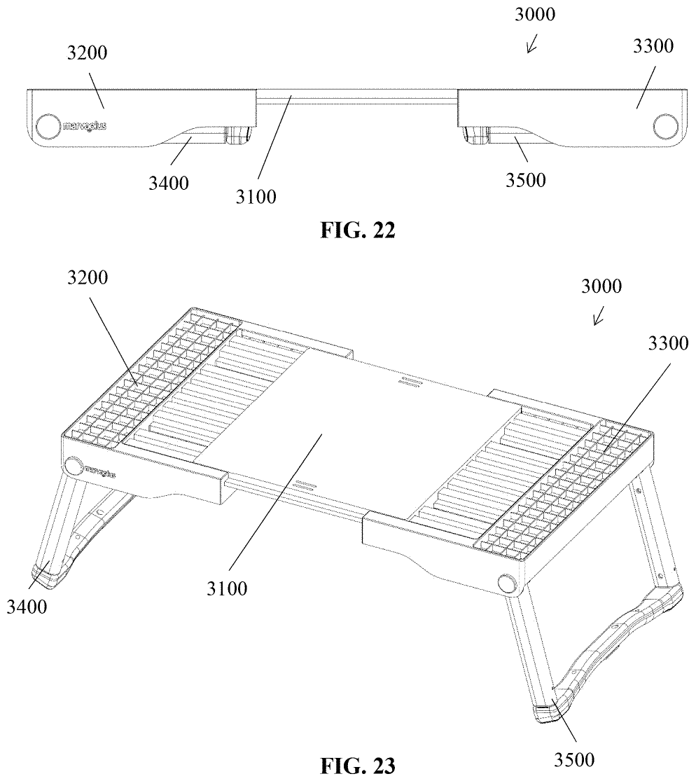

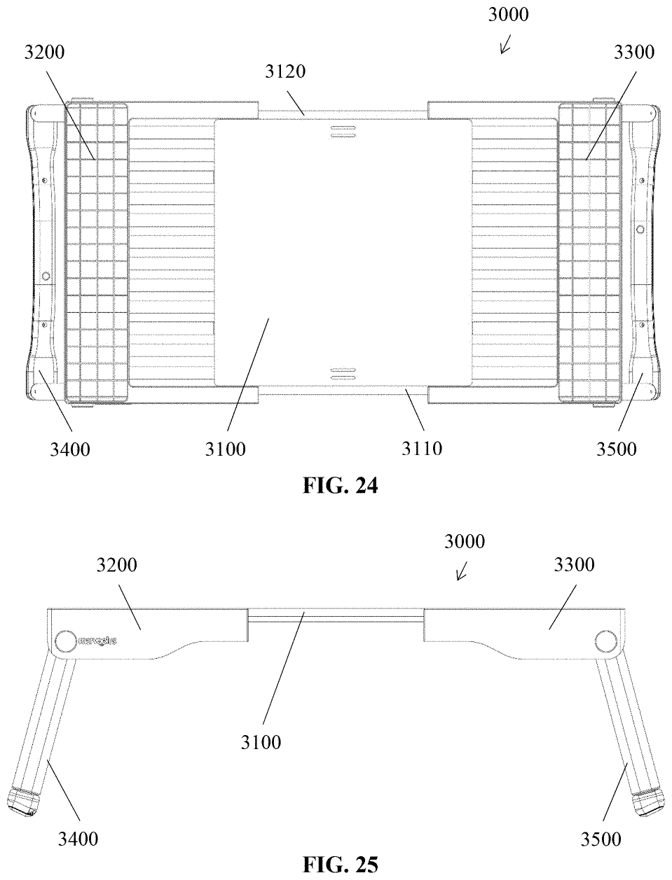

An embodiment of a change table for use with a change table bag is shown in FIGS. 17 to 27. Change table 3000 is depicted to show the expansion and support structure that may be used in a change table incorporated into a change table bag. In some embodiments, change table 3000 may not be incorporated into a change table bag or, alternatively, may be used with any generic bag of suitable dimensions. Change table 2200 of change table bag 2000 includes additional housing structure to enable it to be easily incorporated into the structure of change table bag 2000, such as flaps 2212 and 2214, a handle 2250 on an upper end of change table 2200, and additional housing structure to form a perimeter contour 2260 which correspondent to the contours of the wall structure 2100. Housing structure of change table 2200 is particularly shown in FIG. 14.

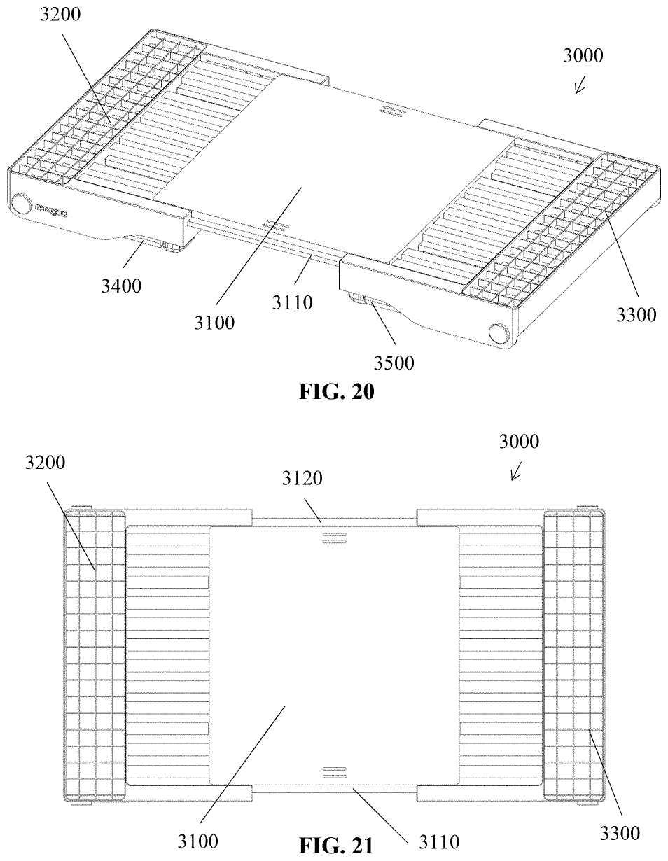

Change table 3000 includes a core 3100, a left extension portion 3200, a right extension portion 3300, a left leg 3400, and a right leg 3500. Left and right extension portion 3200 and 3300 may be moved between a collapsed position resting against core 3100 as shown for example in FIG. 17 and a deployed position drawn out from core 3100 as shown for example in FIG. 20.

Change table 3000 is shown in a portable and collapsed configuration in FIGS. 17 to 19. To deploy change table 3000, a number of deployment steps may be taken by a user. As shown in FIGS. 20 to 22, the user may first draw out left and right extension portions 3200 and 3300 to expand the top surface defined by the change table 3000, which roughly corresponds to the change surface that is to be used as a surface upon which to rest an infant during a diaper change operation. The movement of extension portions 3200 and 3300 is guided by tracks 3110 and 3120 of core 3100. Extension portions 3200 and 3300 are secured to core 3100 by being slidable secured to tracks 3110 and 3120. A system of stops (not shown) is provided on tracks 3110 and 3120 to ensure the extension portions 3200 and 3300 are not drawn off of the tracks. In some embodiments, the top surface of a change table, such as change table 3000, includes a combination of a tightly knit fabric material so that it can be utilized as an occasional infant bed.

A user may then choose to deploy change table support structure. This support structure takes the form of left and right legs 3400 and 3500, each of which is received when not deployed in the change surface structure defined by core 3100, left extension portion 3200, and right extension portion 3300. Left and right legs 3400 and 3500 are lowered as indicated in FIGS. 23 to 25 to allow the change surface to be elevated for ease of use or for sanitary or other reasons.

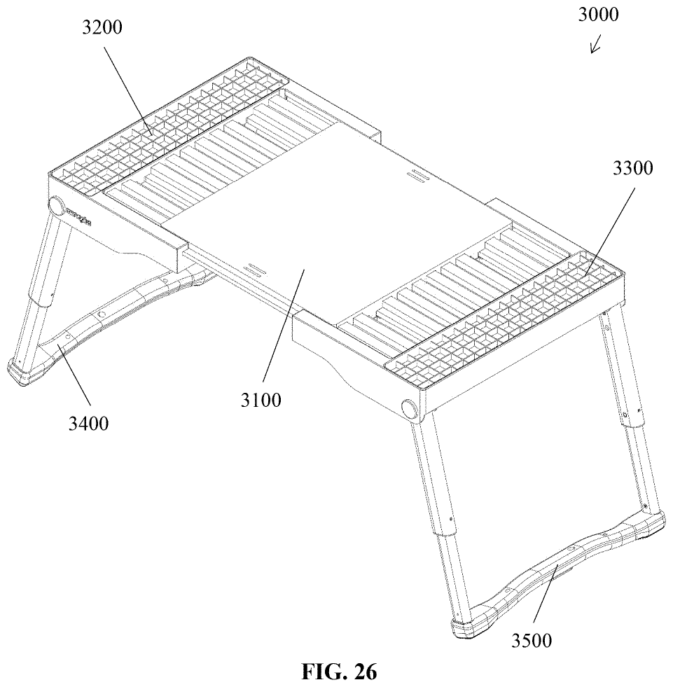

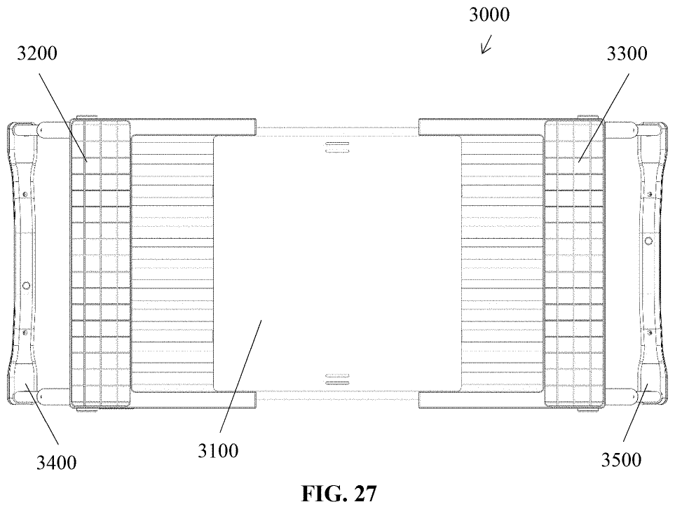

A user may also choose to further raise the change surface by extending left and right legs 3400 and 3500, as indicated in FIGS. 26 and 27. Each of legs 3400 and 3500 are telescoping legs which may be drawn out. Each of legs 3400 and 3500 includes a locking mechanism, the locking mechanism being a push button or pop-out button locking mechanism which is automatically deployed when the telescoping portions are extended to a predetermined length. The locking mechanism may be overcome to retract the telescoping portions of the legs by applying a predetermined force to overcome the pop-out buttons. In some embodiments, legs, such as legs 3400 and 3500, can be further extended to multiple heights, such as to bring the change surface up to a comfortable waist level of an adult or higher.

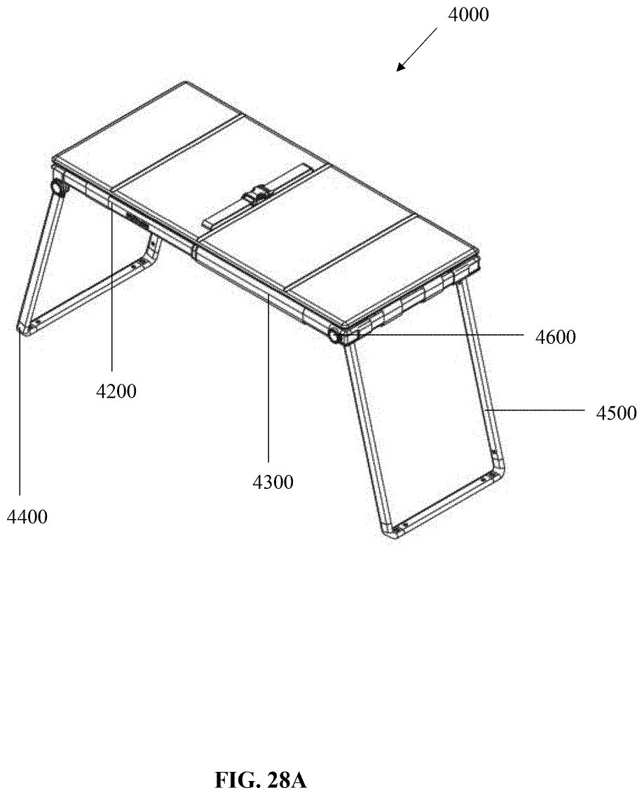

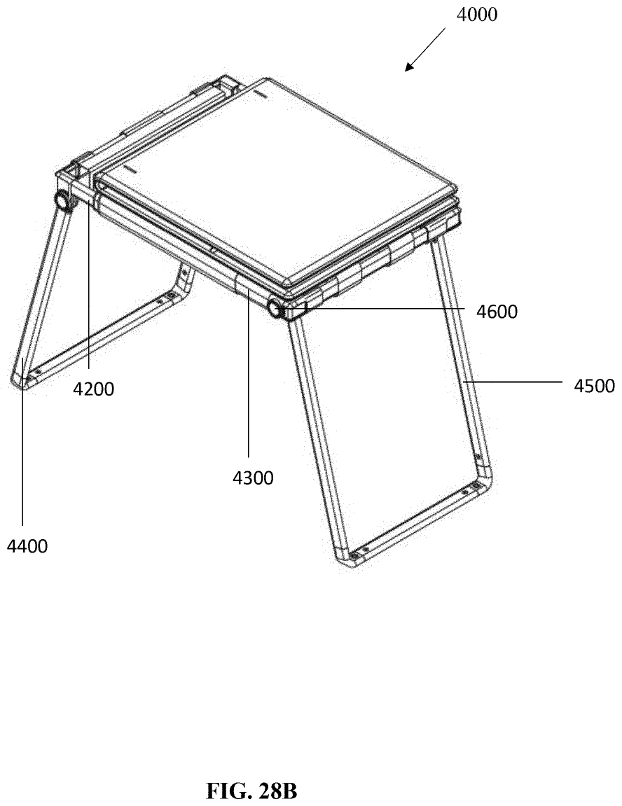

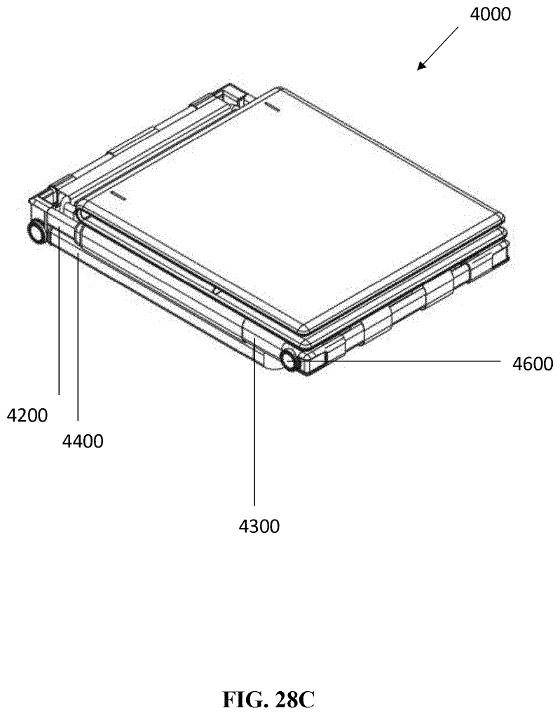

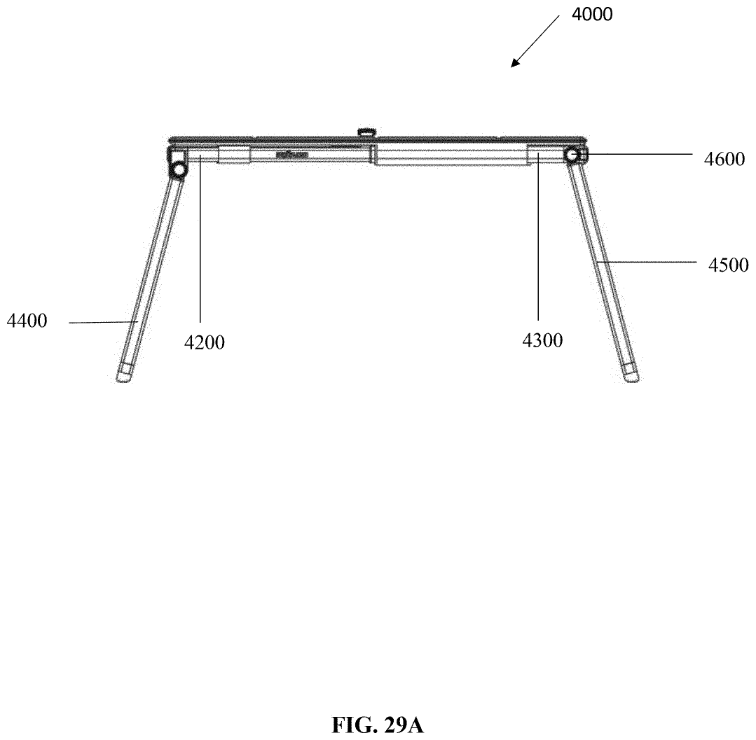

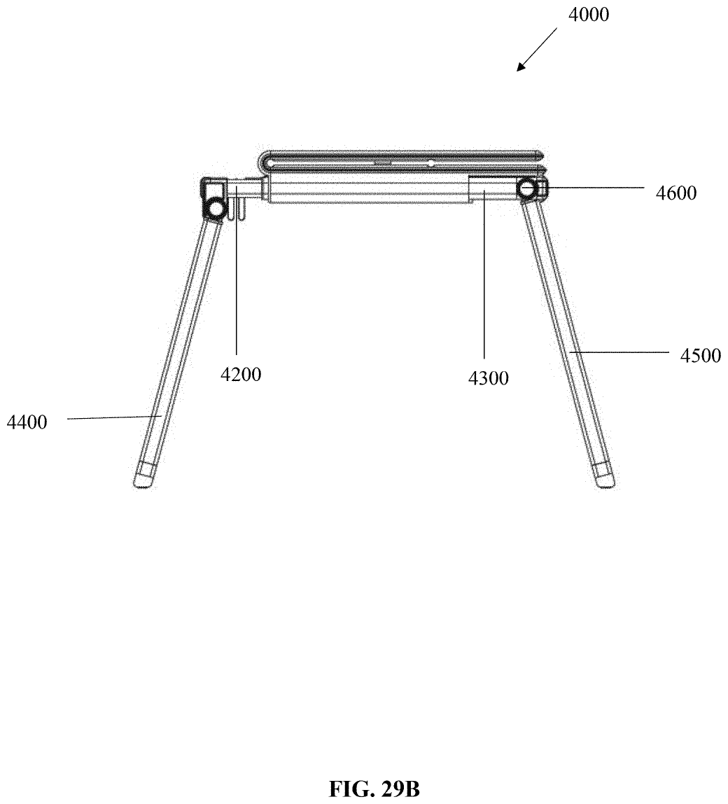

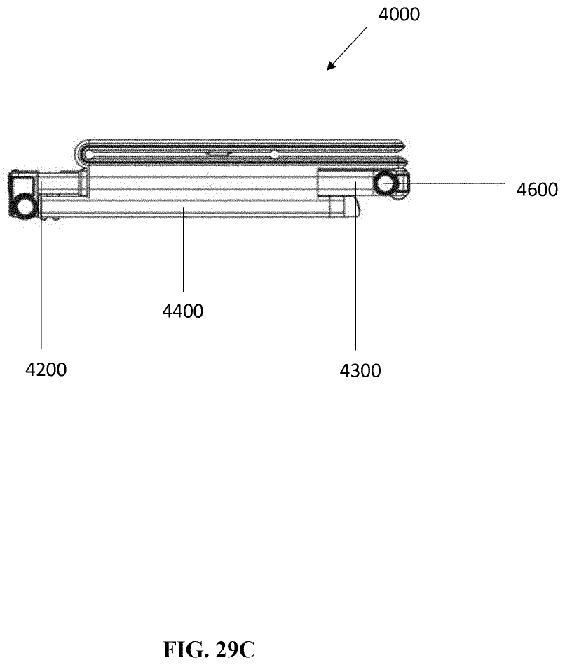

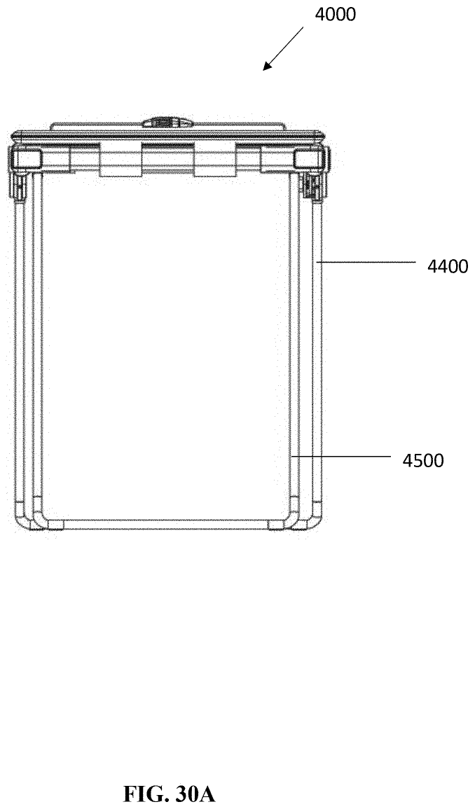

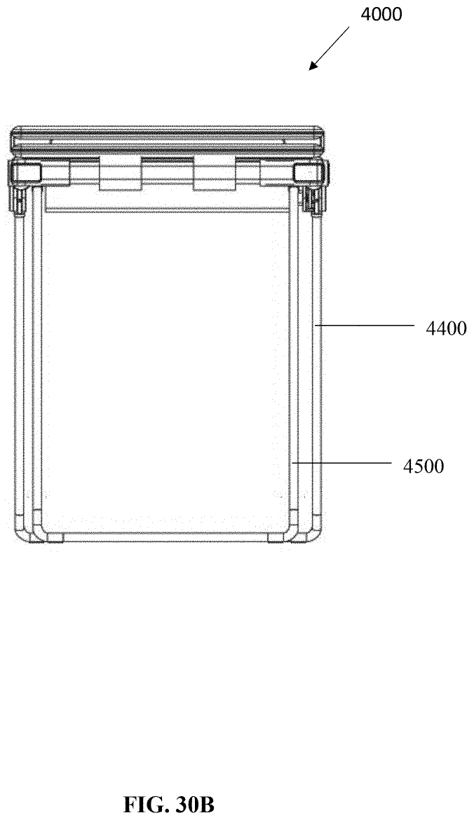

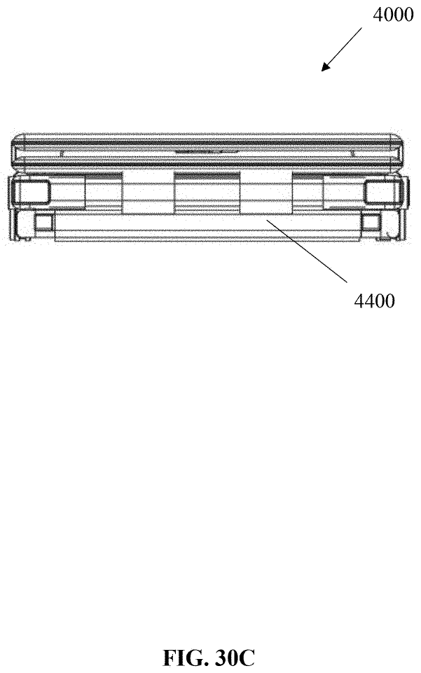

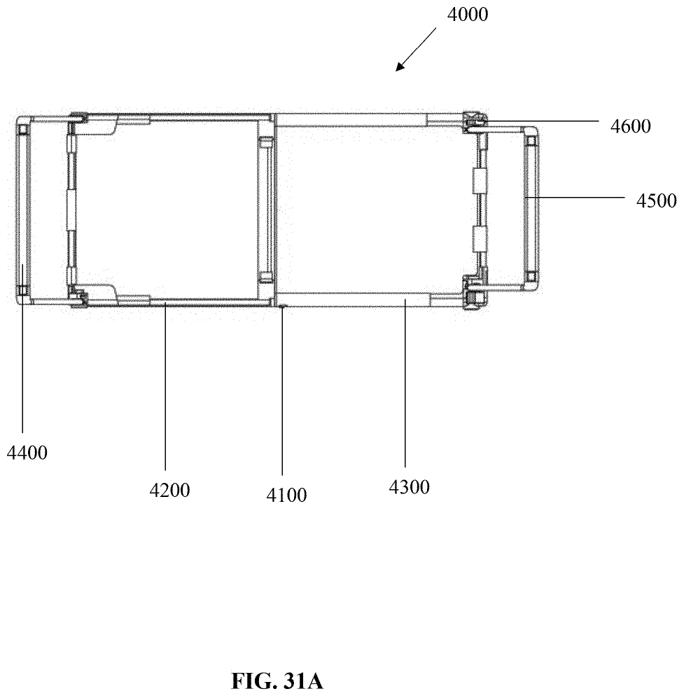

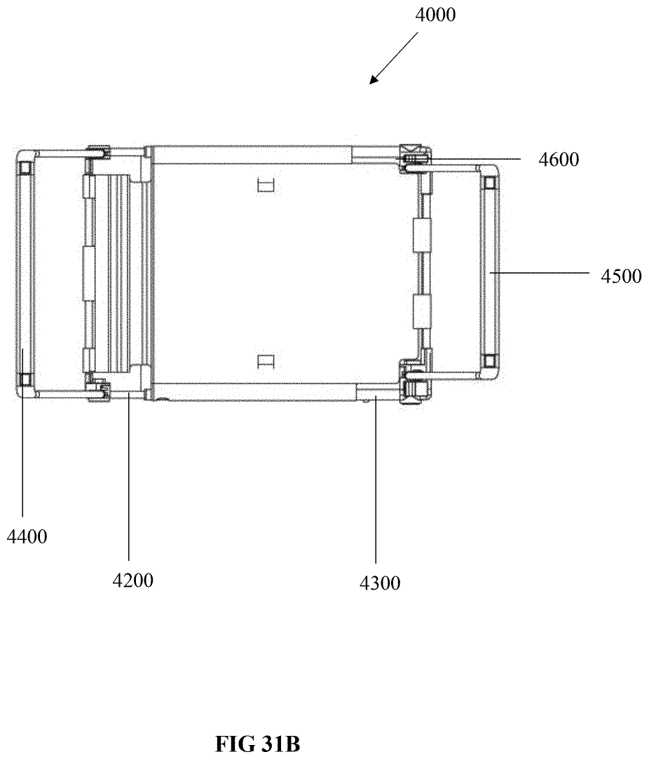

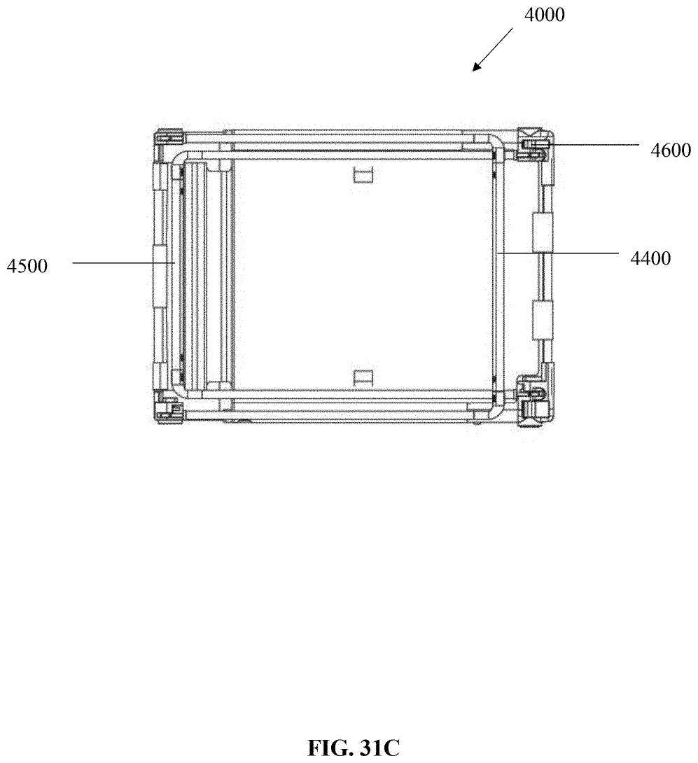

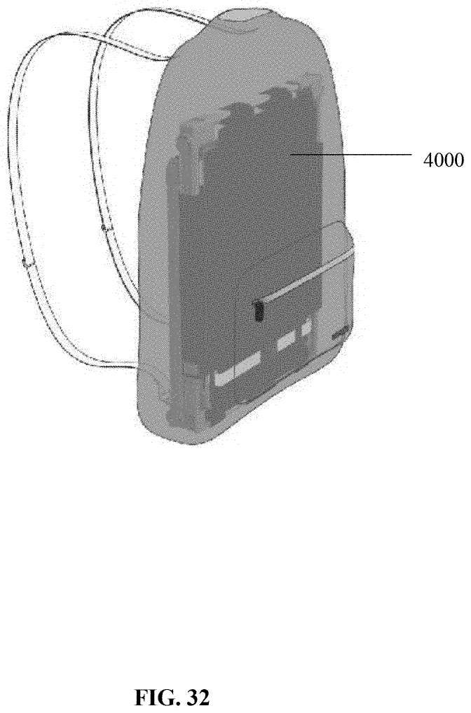

A further embodiment of a change table for use with a change table bag is shown in FIGS. 28 to 32. Change table 4000 is depicted to show the expansion and support structure that may be used in a change table incorporated into a change table bag. In some embodiments, however, change table 4000 may not be incorporated into a change table bag or, alternatively, may be used with any generic bag of suitable dimensions, as shown in FIG. 32.

Change table 4000 includes a left extension portion 4200, a right extension portion 4300, a left leg 4400, and a right leg 4500. Left and right extension portion 4200 and 4300 may be moved between a collapsed position, as shown for example in FIGS. 28C to 31C and a deployed position as shown for example in FIGS. 28A to 31A.

Change table 4000 is shown in a portable and collapsed configuration in FIGS. 28C to 31C. To deploy change table 4000, a number of deployment steps may be taken by a user. The user may first draw out left and right extension portions 4200 and 4300 to expand the top surface defined by the change table 4000, which roughly corresponds to the change surface that is to be used as a surface upon which to rest an infant during a diaper change operation. The movement of extension portions 4200 and 4300 is telescopic. Extension portions 4200 and 4300 are substantially tubular, such that one component can fit within the other and slidably collapse and deploy the top surface defined by change table 4000. When extension portions 4200 and 4300 are deployed, they are secured in placed by a locking mechanism 4100, locking mechanism 4100 being a push button or pop-out button locking mechanism which is automatically deployed when the telescoping portions are extended to a predetermined length. The locking mechanism may be overcome to retract the telescoping portions of the legs by applying a predetermined force to overcome the pop-out buttons. In some embodiments, the top surface of a change table, such as change table 4000, includes a combination of a tightly knit fabric material so that it can be utilized as an occasional infant bed.

A user may then choose to further deploy change table support structure. This support structure takes the form of left and right legs 4400 and 4500, each of which is attached by a hinge 4600 to left extension portion 4200 and right extension portion 4300, respectively. When collapsed, left and right legs 4400 and 4500 are folded and are stored underneath left extension portion 4200 and right extension portion 4300. To deploy change table 4000, left and right legs 4400 and 4500 are extended about hinges 4600 to allow the change surface to be elevated for ease of use or for sanitary or other reasons. When fully extended, hinges 4600 lock, thereby securing left and right legs 4400 and 4500 in an upright position. In some embodiments, left and right legs may be comprised of telescoping legs, in order to further raise the change surface.

Change tables may be formed of a variety of materials, such as plastics, metals, woods, fabrics, or other materials. According to a preferred embodiment, a mixture of aluminum and plastic may be used. In some embodiments, at least a portion of a change table includes a rigid or partially rigid material to form a firm change surface for resting an infant upon. For example, a change tabletop may be constructed from a fabric or canvas mesh, so as to reduce the weight of the product, increase transportability and allow for laundering. In some embodiments, at least a portion of a change table is formed of a semi-malleable plastic or other semi-malleable material, such as to form a housing defining an outer contour which will form part of an outer surface of a change table bag. In some embodiments, a housing of a change table is a fabric-covered semi-malleable plastic to provide uniformity of appearance with a wall structure.

Various embodiments of the invention have been described in detail. Since changes in and or additions to the above-described best mode may be made without departing from the nature, spirit or scope of the invention, the invention is not to be limited to those details but only by the appended claims.

What is claimed is:

1. A disposal bin, comprising:
an outer housing, the outer housing configured to receive an inner bin in a lower portion thereof, the outer housing including a deposit opening in an upper portion thereof through which a disposal item may be introduced into the outer housing; and
an inner bin, the inner bin received in the lower portion of the outer housing, the inner bin defining an inner bin opening in an open upper end thereof for receiving a disposal item when the disposal item is deposited through the deposit opening of the outer housing.

2. The disposal bin of claim 1, further including a drop-through door secured to the inner bin to govern access through the inner bin opening.

3. The disposal bin of claim 2, wherein the deposit opening is governed by an isolation door.

4. The disposal bin of claim 3, wherein the isolation door is triggered to open by an electronic motion sensor located on the disposal bin.

5. The disposal bin system of claim 3, wherein the drop-through door is a trap door style drop-through door biased in a closed position.

6. The disposal bin system of claim 5, wherein the drop-through door is secured to the inner bin with snap lock handles.

7. The disposal bin system of claim 3, wherein the isolation door is in a front panel of the outer housing.

8. The disposal bin system of claim 1, wherein the housing forms a flat top surface.

9. The disposal bin system of claim 8, further including a perimeter light deposited under a rim of the flat top surface.

10. The disposal bin system of claim 9, wherein the perimeter light is activated by triggering an electronic motion sensor located on the disposal bin.

11. The disposal bin of claim 1, wherein the outer housing includes an access opening governed by an access door, configured to permit the inner bin to be moved in and out of the outer housing.

12. The disposal bin of claim 11, further including a slidable toggle locking system to hold the access door closed.

13. The disposal bin of claim 1, further comprising a set of peel-and-stick vinyl skins for application to at least one of the outer housing and the inner bin.

14. The disposal bin of claim 12, further comprising at least one of a sensor system for touch-free operation of a video projector, a speaker, a heater, a file transfer port, and a wireless charging pad.

15. A change table, comprising:
a core and at least one extension portion, the change table being reconfigurable between a retracted, portable configuration and a deployed configuration in which the at least one extension portion of the change table is extended to increase the size of a changing surface.

16. The change table of claim 15, wherein the change table further includes a set of deployable legs which are movable between a retracted position and a deployed position.

17. The change table of claim 16, wherein the set of deployable legs are attached to the change table by locking hinges, whereby the locking hinges allow for the deployable legs to fold and extend about the hinges.

18. The change table of claim 15, wherein the at least one extension portion of the change table is secured in a deployed position by push button or pop-out button locking mechanism.

19. A method of deploying a change table, comprising:
drawing at least one extension portion away from a core to expose a changing surface of the change table; and
deploying a set of legs to support the changing surface above an environmental surface.
